(19) United States
(12) Patent Application Publication
(10) Pub. No.: US 2018/0237853 A1
(43) Pub. Date: Aug. 23, 2018

(54) Methods, Compositions and Kits for Detection of Mutant Variants of Target Genes

(71) Applicant: Yan Wang, San Diego, CA (US)

(72) Inventor: Yan Wang, San Diego, CA (US)

(21) Appl. No.: 15/888,042

(22) Filed: Feb. 4, 2018

Related U.S. Application Data
(60) Provisional application No. 62/460,806, filed on Feb. 19, 2017.

(51) Int. Cl. C12Q 1/6876

(52) U.S. Cl. CPC C12Q 1/6876; C12Q 2600/156

(57) ABSTRACT

Disclosed are methods, compositions and kits for detecting rare changes, mutations or polymorphisms of target nucleic acids in a sample containing an excess of wild-type nucleic acids. The present invention uses universal PCR primers and a universal detection sequence for amplification and detection of different target sequences, allowing detection of the presence of any of multiple target sequences and quantitate the total amount of multiple target sequences in a single-tube reaction. The present method for detecting rare mutants has high specificity and selectivity, is easy to be optimized for different target sequences, and can be adapted to multiplexed automation. Also disclosed are methods for detecting copy number variation of a target gene/chromosome.

### CROSS-REFERENCE TO RELATED APPLICATIONS

This application claims the benefit of U.S. provisional patent application No. 62/460,806, filed Feb. 19, 2017, the content of which is incorporated by reference herein.

### FIELD OF THE INVENTION

This invention relates to methods and technologies in the field of molecular diagnostics, especially relates to methods, compositions and kits for detecting rare mutant variants of target nucleic acids in a sample with an excess of wild-type sequences. Using universal PCR primers and a universal detection sequence, the present invention provides a method for simultaneous detection of up to thousands of target sequences in a single-tube reaction.

### BACKGROUND OF THE INVENTION

The present invention relates to methods, compositions and kits for detecting rare changes, mutations or polymorphisms of target nucleic acids in a sample containing an excess of wild-type nucleic acids. The present invention uses universal PCR primers for amplification and a universal detection sequence for detection of different target sequences, allowing detection of the presence of any of multiple target sequences and quantitate the total amount of multiple target sequences in a single-tube reaction. The present method for detecting rare mutants has high specificity and selectivity, is easy to be optimized for different target sequences, and can be adapted to multiplexed automation.

Many mutant variants of nucleic acids such as Single Nucleotide Polymorphisms (SNPs), insertions/deletions, gene fusions, variants with altered methylation patterns, and copy number variants are implicated in genetic disorders, susceptibility to diseases, predisposition to drug resistance, and progression of diseases, which can be used as important biomarkers for diagnostics, prognostics, and treatments of diseases. Methods and technologies for effectively detecting mutant variants thus play an increasingly important role in clinical applications including diagnosing early phase diseases, detecting prenatal genetic disorders, making prognostic predictions, and designing effective treatment paradigms. In many instances, it is required to detect and quantitate rare disease-associated mutant variants against a high background of wild-type sequences or alternative variants. For example, some low abundance somatic mutations are shown to be associated with cancer prognosis and therapeutic efficacy, and determination of the small fraction of fetal DNAs in the background of maternal DNAs is essential for detecting prenatal genetic disorders. Detection of somatic mutations or fetal DNAs in circulating cell-free DNA samples requires methods of high sensitivity and specificity.

Although having great potentials in clinical applications, detecting rare mutants with high specificity and accuracy in the presence of excess wild-type sequences or alternative variants presents great technical challenges. The amount of wild-type sequences often are 100 times or more higher than that of rare mutant variants in biological samples, resulting in much suppressed detection of the minor mutants. The detection methods may not have enough sensitivity to detect the minor mutants. Various methods involving ligation, hybridization, wild-type blockers, DNA polymerases or nucleases have been attempted to overcome the problem. In addition, the starting materials in clinical samples are limited (e.g. 5-20 ng total DNA) and multiple diagnostic tests are needed, it poses a stringent requirements for high sensitivity and specificity of testing methods.

Allele-specific polymerase chain reaction (AS-PCR) is a widely used method for selectively amplifying and detecting mutant variants (Wu D Y, Ugozzoli L, Pal B K, Wallace R B, Proc Natl Acad Sci USA 1989; 86:2757-2760; Chen X, and Sullivan P F, The Pharmacogeonomics Journal 2003; 3:77-96). AS-PCR uses allele-specific PCR primers complementary to the target polymorphic site of the mutant allele to selectively amplify the mutant variant. The selectivity and specificity of AS-PCR is largely dependent on the fidelity of DNA polymerase that extends primers at a much lower efficiency with a mismatched 3′ end than that with a matched 3′ end. However, exponential PCR amplification makes quick decay of this discriminating power and significant mismatched amplification often occurs. The differentiation ability of this method is also affected by the ratio of wild-type vs. mutant allele and the sequences around the polymorphic base.

Single nucleotide mutation can be detected by a LigAmp assay, which is based on the sequence specificity of DNA ligases to distinguish matching vs. mismatched DNA duplex at the ligation site (Shi C, et al. Nat Methods. 2004 November; 1(2):141-7. and U.S. Pat. No. 8,679,788). In LigAmp assays, two oligonucleotides are hybridized adjacently to a DNA template. Only when the mutant variant of interest is present, the oligonucleotides can be ligated together and detected by real-time PCR. This is a sensitive method for detecting single nucleotide mutations. However, it depends on the sequence specificity of the DNA ligase and non-specific oligonucleotides ligation can lead to false positive detection.

Another method exploits the difference of hybridization strength of matched vs. mismatched DNA duplexes to selectively amplify the amplicon of the mutant allele (US Patent Publication Nos. 2004/0091905, 2015/0315636). This method uses a wild-type blocking probe that is fully complementary to the sequence of polymorphic region of the wild-type allele but not that of the mutant allele to selectively block the amplification of the amplicon of the wide-type allele, and uses a mutant allele-specific reporter to detect the amplicon of the mutant allele. Application of this method needs the polymorphic sequence of the wild-type allele and mutant allele to be known and requires significant optimization for each mutant allele.

However, these methods are not ideal. The production of the amplicon of the mutant allele uses unique primer for each allele, and depends on the exponential amplification using allele-specific primers. The detection of mutant amplicon also depends on the allele-specific reporter probes. As a result, detection of a mutant allele needs significant optimization for each allele. The requirement of specific PCR primers and detection probe for each allele increases the cost for each assay and makes it difficult to develop multiplexed assays since the conditions for different primers and detection probes are less likely to be optimized together. When the amount of mutant alleles is very low, errors due to exponential amplification become more significant and results in false positive results. As such, there is an urgent need for developing reliable and robust technologies that allow sensitive and accurate detection and quantitation of rare mutants of interest, and that can be easily optimized for multiplexed detection of different target sequences and be automated for testing large numbers of samples. The present invention satisfies this need and provides other benefits as well.

### SUMMARY OF THE INVENTION

The present invention provides a method for detecting rare mutant variants of a target gene in a sample containing an excess of wild-type sequence. Using universal PCR primers and a universal detection sequence, the present invention provides a method for simultaneous detection of up to thousands of target sequences in a single-tube reaction. Unlike most of the mutant detection methods that use mutant-specific probes to amplify and detect mutant-specific products, the present invention separates the mutant discrimination reaction from the universal amplification and detection reaction. It first uses an mutant-capturing probe to generate an exonuclease-resistant mutant-specific connection product using a mutant variant, not a wild-type sequence, as a template, and removes the exonuclease susceptible nuclear acids using exonuclease digestion. It then uses universal PCR primers for amplification and a universal detection mechanism for detection of the mutant-specific connection product. This method uses a wild-type blocking probe to block the production of exonuclease-resistant DNA products using the wild-type sequence as a template, further increasing the specificity and selectivity of generating exonuclease-resistant mutant-specific products. This feature is especially important for selective amplification of low frequency mutant variants (e.g. <1% of the total target gene population). The exonuclease clean-up step is essential for creating a robust and efficient detection method with little background noise and interference. This method also provides a means for linear amplification of the mutant-specific connection products before subjecting them to PCR amplification and detection. This method also provides a method for selectively amplifying mutant variants with unknown mutations.

A salient feature of this method is to use a pair of universal PCR primers and a universal detection probe to detect multiple target sequences in a single reaction. To detect different mutant variants or target genes, only the target-specific portions of the capturing probes need to be customized while the universal primer recognition and universal detection portion can be kept the same for different capturing probes. In this method, different mutants of the same gene and mutants of different genes can be amplified and detected under similar conditions, allowing easy optimization and automation of the detection process and significant reduction of measurement variation for different target sequences. Each mutant-specific probe or each group of mutant-specific probes can also be linked to a distinct detection probe (e.g. different fluorescent dyes), providing an approach for multiplexed detection of different target sequences. Although this method has the feature of detecting mutant variant of a target gene, it can be applied to detect any target sequence whether it is a mutant or wild-type sequence. By using different target-specific sequences while using the same universal PCR primers and universal detection sequence, it can be used to detect the presence of any one of a plurality of target sequences or to quantitate the total amount of a plurality of target sequences in a nucleic acid sample, which provides significant saving in sample usage, labor and cost. For example, it can be used to simultaneously detect the presence of any of multiple gene mutations associated with a single disease. It can be used to detect the amount of hundreds or thousands of gene loci on a target chromosome for determination of copy number variation.

A method for detecting a target sequence in a nucleic acid sample containing, comprising the steps of:
a) providing a target-capturing probe containing a target-specific portion and a universal primer binding and a universal detection portion;
b) selecting a target-capturing probe from
   i. a single polynucleotide, from 5′ to 3′ end sequentially comprising a 5′-target-specific sequence (5′-TSS), a forward universal primer binding sequence (Forward-UPBS), a breakable site, a reverse universal primer binding sequence (Reverse-UPBS) and a 3′-target-specific sequence (3′-TSS), having a universal detection sequence inserted between the Forward-UPBS and the 5′-TSS or the Reverse-UPBS and the 3′-TSS, or
   ii. two polynucleotides, comprising a first polynucleotide that comprises an exonuclease-resistant 5′end, a Reverse-UPBS, and a 3′-TSS at the 3′ end, and a second polynucleotide that comprises a 5′-TSS at the 5′ end, a Forward-UPBS, and an exonuclease-resistant 3′ end, having a universal detection sequence inserted between the Forward-UPBS and the 5′-TSS or the Reverse-UPBS and the 3′-TSS;
c) contacting the target-capturing probe with the nucleic acid sample under conditions where the 3′-TSS and the 5′-TSS of the target-capturing probe anneal to the target sequence in the sample to form a duplex nucleic acid;
d) performing a target-selective connection wherein the 3′-TSS and the 5′-TSS of the target-capturing probe are connected to form a linear or circular exonuclease-resistant target-specific connection product using the target sequence as a template;
e) Optionally, using an exonuclease to digest susceptible nucleic acids, and after the enzymatic digestion, inactivating the exonuclease and/or purifying the exonuclease-resistant target-specific connection products;
f) breaking the circular exonuclease-resistant target-specific connection products, if present, at the breakable site to obtain linear target-specific connection products;
g) amplifying the linear target-specific connection products or the linear exonuclease-resistant target-specific connection products by a polymerase chain reaction (PCR) using universal PCR primers recognizing the universal primer binding sequences or the complement thereof; and
h) detecting or quantitating the linear target-specific connection products or the linear exonuclease-resistant target-specific connection products as a measurement of the target sequences using a sequence-specific reporter probe recognizing the universal detection sequence or the complement thereof.

The target-capturing probe can be a single polynucleotide or two polynucleotides, which are designed to generate an exonuclease-resistant target-specific connection product selectively using the target sequence as a template. The exonuclease-resistant target-specific connection product is then separated and purified while the other nucleic acids, including unligated target-capturing probes, single stranded and double stranded DNA sequences from the nucleic acid sample, and non-specific ligation products, are digested by an exonuclease. The exonuclease used in this method, which can be a single enzyme or multiple enzymes, should have 3′->5′ and 5′->3′ exonuclease activity, and can digest both ssDNA and dsDNA. The Forward-UPBS and the Reverse-UPBS are universal primer binding sequences that can be used in different target-capturing probes for PCR amplification of different target sequences. The PCR primers are designed such that the PCR amplicon encompasses the connected 3′-TSS and 5′-TSS. The breakable site comprises a chemical moiety that is susceptible to photo, enzymatic or chemical cleavage, and is used to convert circular target-specific connection product into linear target-specific connection product. The universal detection sequence is a predesigned sequence that can hybridize with a sequence-specific reporter probe to generate a detectable signal. The reporter probe can be, for example, a Taqman® probe (Thermo Fisher Scientific, Waltham, Mass.), a Scorpion® reporter probe (Sigma-Aldrich, St. Louis, Mo.) or a Molecular Beacon probe.

In some embodiment, the target-capturing probe is used to capture a mutant variant of a target gene and is thus called as a mutant-capturing probe. The mutant-capturing probe is used to capture mutant sequences and form mutant-specific connection products in the step d) of the above method. The mutant-capturing probe selectively captures the mutant variant over the wild-type sequence of the target gene, allowing detection of rare mutant alleles in the presence of excess wild-type sequences.

In some embodiment, the method further comprises repeating the step c) and d) multiple times for linear amplification of the mutant-specific connection product. The steps can be repeated for 1-100 times depending on the abundance of the mutant variant in the sample. In an ideal situation, one mutant-specific connection product corresponds to one copy of the mutant variant in the sample. This linear amplification can increase detection sensitivity and detection limit for low frequency mutant variants.

In some embodiment, the amplification and the detection of the linear mutant-specific connection products or the linear exonuclease-resistant mutant-specific connection products happen concurrently.

In some embodiment, a blocking probe that is complementary to a sequence of a wild-type counterpart region of the mutated region in the mutant variant is added in step c), and wherein the blocking probe blocks the formation of an exonuclease-resistant connection product using the wild-type sequence as a template. Using the blocking probe can greatly decrease the production of connection products based on wild-type sequences, thus greatly lowering the occurrence of false positives and background noises, and increasing detection sensitivity and specificity.

In some embodiment, the 3′-TSS and the 5′-TSS of a mutant-capturing probe anneal to a locus at or close to a mutated sequence of the target gene. In some embodiment, the 3′-TSS and/or 5′-TSS of a mutant-capturing probe comprise a sequence complementary to a portion of the mutant sequence. In some embodiment, the 3′-TSS of a mutant-capturing probe contains a mutated nucleotide at its 3′ end and/or the 5′-TSS of the mutant-capturing probe contains a mutated nucleotide at its 5′ end.

In some embodiment, a plurality of mutant-capturing probes comprising different target-specific sequences and the same primer binding sequence and the same detection sequence are used to detect the presence of a plurality of mutant variants of target genes. The plurality of mutant-capturing probes may comprise target-specific sequences complementary to different mutant variants of the same target gene. The plurality of mutant-capturing probes may comprise target-specific sequences complementary to mutant variants of different target genes. In some embodiment, a plurality of mutant-capturing probes are divided into multiple groups, wherein members of each group have different target-specific sequences, share a universal detection sequence that is specific for the group, and share the same universal primer binding sequences among all the members of different groups.

In some embodiment, the 3′-TSS of a mutant-capturing probe contains a mutated nucleotide at its 3′ end and/or the 5′-TSS of the mutant-capturing probe contains a mutated nucleotide at its 5′ end. In some embodiment, the 3′-TSS and the 5′-TSS of a mutant-capturing probe anneal adjacently at the mutated region of the mutant variant; and the exonuclease-resistant mutant-specific connection product is formed by direct ligation of the 3′-TSS and the 5′-TSS in the duplex formed by the mutant-capturing probe and the mutant sequence.

In some embodiment, the 3′-TSS and the 5′-TSS of a mutant-capturing probe anneal to the target gene to form a DNA duplex with a single nucleotide gap at the locus of a mutated nucleotide. The exonuclease-resistant mutant-specific connection product is formed by extending the 3′-TSS with the nucleotide complementary to the mutated nucleotide using a DNA polymerase and subsequent DNA ligation to connect the extended 3′-TSS and the 5′-TSS (single nucleotide extension (SNE) and ligation). The DNA polymerase is preferably a thermostable DNA polymerase that lacks 5′->3′ exonuclease activity and does not express strand displacement activity. In some embodiment, only one type of matching nucleotide is provided during the extension reaction. In some embodiment, all the four types of nucleotides and a wild-type blocking probe are provided during the extension reaction.

In some embodiment, the 3′-TSS and the 5′-TSS of a mutant-capturing probe anneal to the target gene to form a DNA duplex with a multi-nucleotide gap that overlaps with the mutated locus, and the exonuclease-resistant mutant-specific connection product is formed by a polynucleotide extension (PNE) from the 3′ end of the 3′-TSS using a DNA polymerase and subsequent DNA ligation to connect the extended 3′-TSS and the 5′-TSS.

In some embodiment, the 3′ end of the 3′-TSS is complementary to a mutated nucleotide, and DNA polymerases can only extend the 3′ end of the 3′-TSS using the mutant variant as a template. Preferably, the DNA polymerase is a thermostable DNA polymerase that lacks 3′->5′ and 5′->3′ exonuclease activity and does not express strand displacement activity.

In some embodiment, the locus of the mutated region is known but the identity of mutated nucleotides is unknown. The 3′-TSS and the 5′-TSS of a mutant-capturing probe are designed to be complementary to the region upstream and downstream of the mutated region. And the 3′-TSS and the 5′-TSS anneal to the target gene to form a DNA duplex with a multi-nucleotide gap that overlaps with the mutated locus. A non-extendable blocking probe that is complementary to the wild-type counterpart region of the mutated region is added in step c), wherein the non-extendable blocking probe blocks the annealing of the 3′-TSS and/or the 5′-TSS to the wild-type sequence, and/or extension of the 3′-TSS by DNA polymerases using the wild-type sequence as a template. The blocking probe is selected to have high binding strength with the complementary sequence of the wild-type gene and a significant loss in binding strength when a putative mutation is introduced into the complementary sequence. In some embodiment, the sequence of the non-extendable blocking probe overlaps with the 3′-TSS and/or 5′-TSS. In some embodiment, the non-extendable blocking probe comprises modified nucleotides. The modified nucleotide can be a peptide nucleic acid, a locked nucleic acid, or a nucleotide linked to a minor groove binder.

In some embodiment, this method is used to detect a mutant variant that has mutations such as nucleotide substitution, deletion, insertion, gene fusion or any combination thereof when compared to the wild-type sequence.

In some embodiment, the nucleic acid sample is an RNA sample. The 3′-TSS and 5′-TSS anneal to the target RNA to form a RNA-DNA duplex. To connect DNA strands in a RNA-DNA duplex, a RNA-templated DNA ligation can be carried out using, for example, T4 DNA ligase that can ligate adjacent DNA strands in a DNA-RNA duplex (US Patent Publication No. 20100184618; Nilsson M, et al. Nucleic Acids Res. 2001, 29(2): 578-581). In some embodiment, a reverse transcriptase is used to extend the 3′-TSS using a target RNA as a template. The extension product is then connected to form an exonuclease-resistant mutant-specific connection product using RNA-templated DNA ligation. In some embodiment, the nucleic acid sample is an RNA sample that is first converted to a cDNA sample and then subjected to the same method as described herein.

In some embodiment, the method is used to detect the methylation of a target gene. The unmethylated and the methylated form of the target gene is considered as the wild-type and mutant gene, respectively. The target gene is modified by converting all the non-methylated cytosine to uracil and the methylated cytosine remains unchanged as a cytosine. The methylation of the target gene can be detected by the presence of the putative U->C mutation at the putative methylation site in the modified target gene.

In some embodiment, the method is used to detect a gene fusion mutant having a first gene portion, a fusion locus, and a second gene portion. In some embodiment, the 3′-TSS of the mutant-capturing probe is complementary to a region of the first gene and the 5′-TSS of the mutant-capturing probe is complementary to a region of the second gene. In some embodiment, the 3′-TSS comprises a sequence complementary to a region having portions of the first and the second gene and the 5′-TSS comprises a sequence complementary to a region of the second gene. In some embodiment, the 3′-TSS comprises a sequence complementary to a region of the first gene and the 5′-TSS comprises a sequence complementary to a region having portions of the first and the second gene. In some embodiment, the 3′-TSS and the 5′-TSS adjacently anneal to the gene fusion mutant. In some embodiment, the 3′-TSS and the 5′-TSS anneal to the gene fusion mutant to form a duplex with a single- or multi-nucleotide gap. Additionally, a blocking probe complementary to a wild-type sequence encompassing the gene fusion locus is added in step c).

In some embodiment, the gene fusion mutant has a known first gene portion, a known fusion locus, and an unknown second gene portion. To detect fusion mutants with unknown fusion sequence, the nucleic acids in the sample are first linked to a predesigned sequence tag on both ends or the 5′ end. The 3′-TSS of the mutant-capturing probe is designed to comprise a sequence complementary to a region upstream to the fusion locus in the first gene, the 5′-TSS is designed to comprise a sequence complementary to the 5′ sequence tag, and a blocking probe complementary to a wild-type sequence encompassing the gene fusion locus is added in step c), which allows selective formation of mutant-specific connection product based on fusion mutants. Using a universal PCR primers and a universal detection sequence, this method allows selective amplification and simultaneous detection of all the unknown fusion mutants having a particular fusion junction.

In some embodiment, the method is used to detect a wild-type sequence of a target gene. The 3′-TSS and the 5′-TSS of the target-capturing probe are connected by ligation or extension-ligation to form linear or circular exonuclease-resistant target-specific connection product using the wild-type target sequences as a template, which can be amplified and detected using the universal PCR primers and the universal detection sequence. In some embodiment, a plurality of target-capturing probes comprising different target-specific sequences with the same universal primer binding sequences and the same universal detection sequence are used to simultaneously detect a plurality of target sequences.

In some embodiment, the method is used to detect copy number variation of a target gene/chromosome by comparing the total copy number of multiple gene loci on the target gene/chromosome to those of a reference gene/chromosome. The method uses two groups of target-capturing probes comprising a first group having a 3′-TSS and a 5′-TSS complementary to sequences of different gene loci on the target gene/chromosome and a second group having a 3′-TSS and a 5′-TSS complementary to sequences of different gene loci on the reference gene/chromosome. Both groups share the same primer binding sequences but have distinct detection sequences. The ratio of the total copy number of different gene loci on the target gene/chromosome vs. the reference gene/chromosome is used to determine the presence of a copy number variation.

In some embodiment, this method can be used to detect the copy number variation of the same gene in different samples. In this case, the target-capturing probes have the same target-specific portions and use different detection sequences for different samples. Once the target specific connection products for different samples are separately captured using target-capturing probes with different detection sequences, they are pooled together and the gene copy number for different samples can be detected by the respective sequence-specific reporter probes.

In some embodiment, the present invention provides a kit for detecting a mutant variant of a target gene in a nucleic acid sample containing an excess of wild-type sequences, comprising:
a) a mutant-capturing probe containing a target-specific portion and a universal primer binding and a universal detection portion, comprising
   i. a single polynucleotide, from 5′ to 3′ end sequentially comprising a 5′-target-specific sequence (5′-TSS), a forward universal primer binding sequence (Forward-UPBS), a breakable site, a reverse universal primer binding sequence (Reverse-UPBS) and a 3′-target-specific sequence (3′-TSS), having a universal detection sequence inserted between the Forward-UPBS and the 5′-TSS or the Reverse-UPBS and the 3′-TSS, or
   ii. two polynucleotides, comprising a first polynucleotide that comprises an exonuclease-resistant 5′ end, a Reverse-UPBS, and a 3′-TSS at the 3′ end, and a second polynucleotide that comprises a 5′-TSS at the 5′ end, a Forward-UPBS, and an exonuclease-resistant 3′ end, having a universal detection sequence inserted between the Forward-UPBS and the 5′-TSS or the Reverse-UPBS and the 3′-TSS;
b) a DNA ligase;
c) a sequence-specific reporter probe recognizing the universal detection sequence;
d) an exonuclease;
e) optionally a DNA polymerase; and
f) an instruction manual.

In some embodiment, the 3′-TSS contains a mutated nucleotide at its 3′ end and/or the 5′-TSS contains a mutated nucleotide at its 5′ end, and the 3′-TSS and the 5′-TSS anneal adjacently at the mutated region of the mutant variant.

In some embodiment, the 3′-TSS and the 5′-TSS anneal to the target gene to form a DNA duplex with a single nucleotide gap, wherein the single nucleotide gap is at the locus of a mutated nucleotide.

In some embodiment, the 3′-TSS and the 5′-TSS anneal to the target gene to form a DNA duplex with a multi-nucleotide, wherein the multi-nucleotide gap overlaps with a mutated region.

In some embodiment, the kit further comprises a non-extendable blocking probe that is complementary to a sequence of the wild-type counterpart region of the mutated region in the mutant variant.

In some embodiment, the kit comprises a plurality of mutant-capturing probes, each having a pair of target-specific sequences complementary to regions of respective target gene sequence close to or at a mutated region, and wherein all the mutant-capturing probes have the same Forward-UPBS and Reverse-UPBS and the same detection sequence. In some embodiment, the kit comprises a plurality of mutant-capturing probes comprising target-specific sequences complementary to sequences of different mutant variants of the same target gene. In some embodiment, the kit comprises a plurality of mutant-capturing probes comprising target-specific sequences complementary to sequences of mutant variants of different target genes. In some embodiment, the kit comprises different groups of mutant-capturing probes, each group having a distinct detection sequence coupled to a different detection signal.

In some embodiment of the kit, the exonuclease comprise one or more enzymes that possess 5′->3′ and 3′->5′ exonuclease activity, and can digest ssDNA and dsDNA.

In some embodiment, the DNA polymerase lacks 5′->3′ exonuclease activity and does not express strand displacement activity.

In some embodiment, the present invention provides a kit for detecting and quantitating a target gene in a nucleic acid sample, comprising:
a) a target-capturing probe containing a target-specific portion and a universal primer binding and a universal detection portion, comprising
   i. a single polynucleotide, from 5′ to 3′ end sequentially comprising a 5′-TSS, a Forward-UPBS, a breakable site, a Reverse-UPBS and a 3′-TSS, having a universal detection sequence inserted between the Forward-UPBS and the 5′-TSS or the Reverse-UPBS and the 3′-TSS or
   ii. two polynucleotides, comprising a first polynucleotide that comprises an exonuclease-resistant 5′ end, a Reverse-UPBS, and a 3′-TSS at the 3′ end, and a second polynucleotide that comprises a 5′-TSS at the 5′ end, a Forward-UPBS, and an exonuclease-resistant 3′ end, having a universal detection sequence inserted between the Forward-UPBS and the 5′-TSS or the Reverse-UPBS and the 3′-TSS;
b) a DNA ligase;
c) a reporter probe specific for the detection sequence;
d) an exonuclease;
e) optionally a DNA polymerase; and
f) an instruction manual.

In some embodiment, the kit provides a plurality of target-capturing probes, each having a pair of target-specific sequences complementary to regions of respective target gene sequences, and all the target-capturing probes have the same Forward-UPBS and Reverse-UPBS and the same detection sequence. In some embodiment, the plurality of target-capturing probes comprises target-specific sequences complementary to different regions of a target gene or a target chromosome. In some embodiment, the plurality of target-capturing probes is divided into multiple groups, each group having a distinct detection sequence coupled to a different detection signal. In some embodiment, the target-capturing probes are divided into a first group that are specific for multiple gene loci on a target gene/chromosome, and a second group that are specific for multiple gene loci on a reference gene/chromosome. The kit can be used for detect copy number variation of the target gene/chromosome as compared to that of the reference gene/chromosome.

### BRIEF DESCRIPTION OF DRAWINGS

FIG. 1. Structures of two types of target-capturing probes. A, a target-capturing probe comprising a single polynucleotide. B, a target-capturing probe comprising two polynucleotides.

FIG. 2. Working diagram of detecting mutant variants using a mutant-capturing probe of a single polynucleotide.

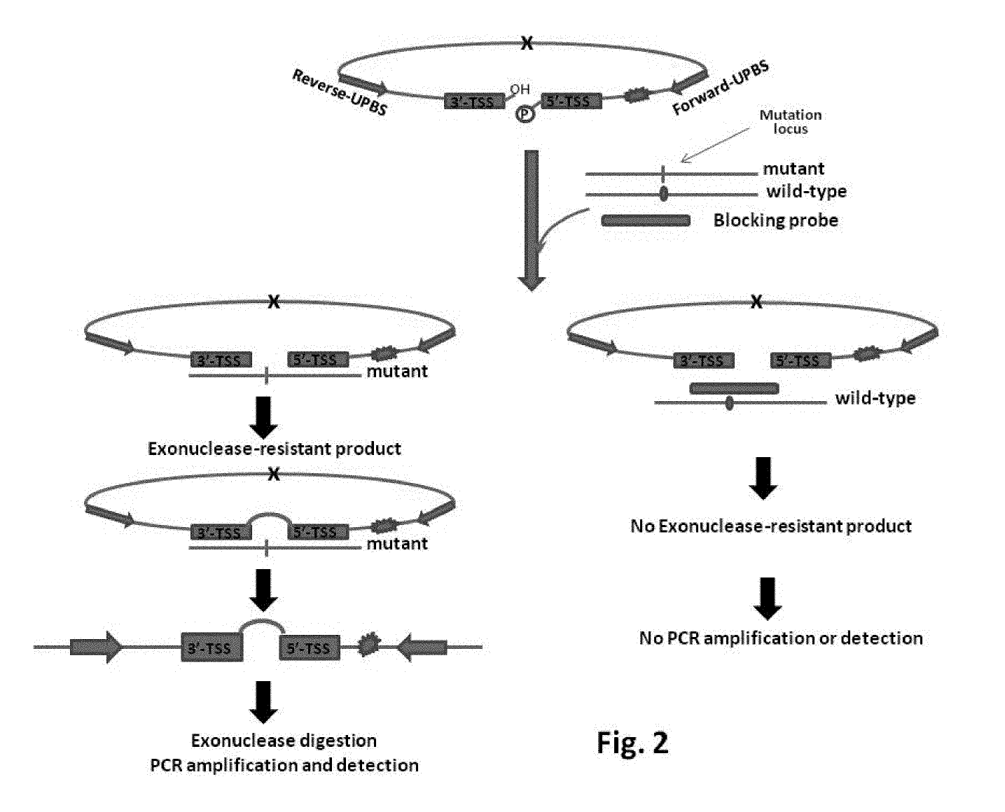

FIG. 3. Working diagram of detecting mutant variants using a mutant-capturing probe of two polynucleotides.

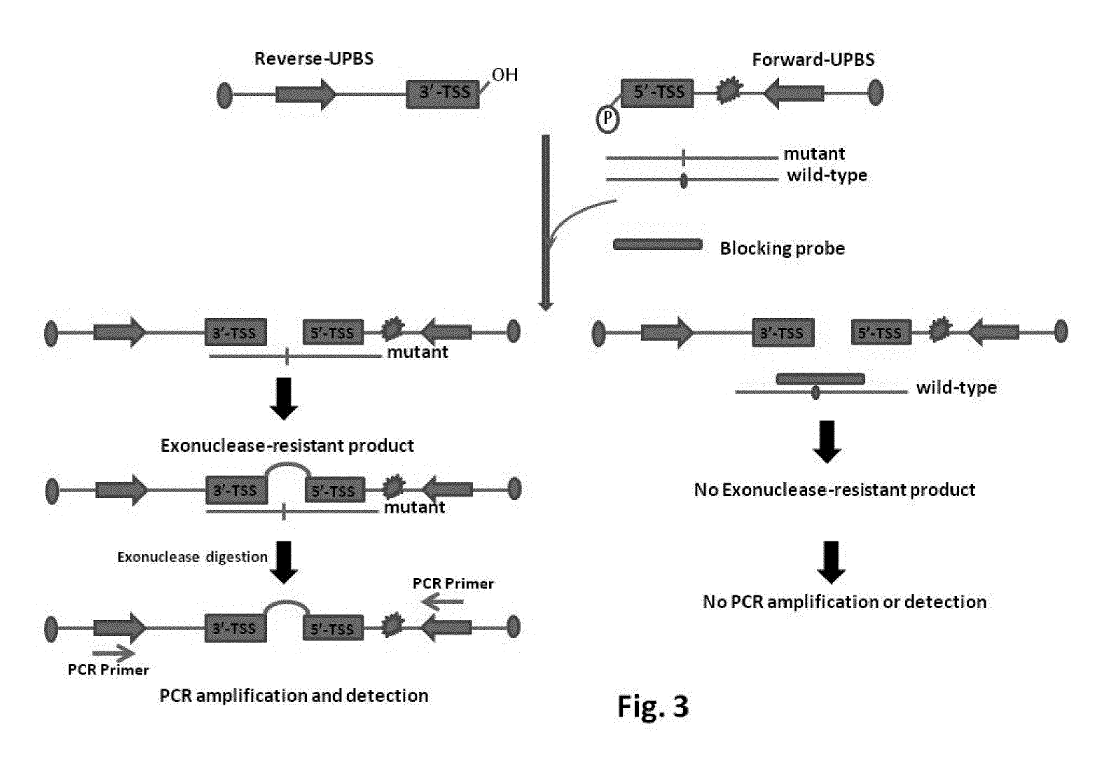

FIG. 4. Examples of mutant-selective connection to make exonuclease-resistant mutant-specific connection product using a mutant variant as a template. A, selection by direct ligation. B, selection by single nucleotide extension (SNE) and ligation.

FIG. 5. Examples of mutant-selective connection to make exonuclease-resistant mutant-specific connection product using a mutant variant as a template. A, selection by using mutant-specific polynucleotide extension (PNE) and ligation. B, selection by using a wild-type blocking probe.

FIG. 6. A PCR-based method for making a capturing probe.

FIG. 7. An extension-ligation method for making a capturing probe.

### DETAILED DESCRIPTION OF THE INVENTION

### Definitions

Unless otherwise defined, all technical and scientific terms used herein have the same meaning as commonly understood by one of the ordinary skill in the art to which this invention belongs.

The term “a” and “an” and “the” as used to describe the invention, should be construed to cover both the singular and the plural, unless explicitly indicated otherwise, or clearly contradicted by context. Similarly, plural terms as used to describe the invention, for example, nucleic acids, nucleotides and DNAs, should also be construed to cover both the plural and the singular, unless indicated otherwise, or clearly contradicted by context.

The term “nucleic acid sample” as used herein, refers to a population of DNA or RNA sequences obtained from any sources. For example, a nucleic acid sample may be prepared from cells, tissues, organs, any other biological and environmental sources. Particularly, a nucleic acid sample may be prepared from a patient's tissue, a body fluid, or a cell sample such as urine, lymph fluid, spinal fluid, synovial fluid, serum, plasma, saliva, skin, stools, sputum, blood cells, tumor cells/tissues, organs, and also samples of in vitro cell culture constituents, which can be used for molecular diagnostic and prognostic purpose. A nucleic acid sample may comprise circulating cell-free DNA, genomic sequences, subgenomic sequences, chromosomal sequences, PCR products, cDNA sequences, smRNA sequences, rRNA sequences, mRNA sequences or whole transcriptome sequences. The DNA or RNA sequences can be linked to preselected sequence tags on one or both ends. The sequence tags are predesigned sequences that are non-complementary to all the sequences in the nucleic acid sample.

The term “target gene”, as used herein, refers to a region or locus of DNA or RNA that is of particular interest to the user, for example, it is related to a disease or drug resistance. A target gene may be a DNA coding region of a protein, a regulatory region of a gene, and a region of an mRNA, an smRNA, a miRNA or an rRNA. The target gene usually has various forms in terms of the nucleic acid sequence. The most common and prevalent form in a population is the “wild-type sequence” or “wild-type gene”. The other forms having mutations relative to the wild-type sequence are considered “mutant variants”. The mutations include, for example, nucleotide substitutions, insertions, deletions, gene fusions, and any combination thereof. The location where the sequence divergence occurs between a mutant variant and a wild-type sequence is a mutated region. A mutated region, as used herein, refers to a continuous section of a sequence that includes the actual locus of nucleotide substitution, insertion, deletion, and gene fusion. A mutant variant can have more than one mutated regions compared to a wild-type sequence.

The term “target-capturing probe”, as used herein, refers to a ssDNA or dsDNA probe comprising a target-specific portion and a universal primer binding and universal detection portion, which can used to anneal to a target sequence and selectively make an exonuclease-resistant target-specific connection product that can be amplified and detected using a universal primer binding sequence and a universal detection sequence. In some embodiment, the target capturing probe comprises a single polynucleotide, from 5′ to 3′ end sequentially comprising a 5′-target-specific sequence (5′-TSS), a forward universal primer binding sequence (Forward-UPBS), a breakable site, a reverse universal primer binding sequence (Reverse-UPBS) and a 3′-target-specific sequence (3′-TSS), having a universal detection sequence inserted between the Forward-UPBS and the 5′-TSS or the Reverse-UPBS and the 3′-TSS. In some embodiment, the target-capturing peptide has two polynucleotides, which comprise a first polynucleotide that comprises an exonuclease-resistant 5′ end, a Reverse-UPBS, and a 3′-TSS at the 3′ end, and a second polynucleotide that comprises a 5′-TSS, a Forward-UPBS, and an exonuclease-resistant 3′ end, having a universal detection sequence inserted between the Forward-UPBS and the 5′-TSS or the Reverse-UPBS and the 3′-TSS. The 5′- and 3′-TSS of the target-capturing probe anneal to the same strand of the target sequence and can be connected via direct ligation or extension/ligation to form an exonuclease-resistant connection product only when the target sequence is present in the sample.

The target-capturing probes can be used to capture a plurality of target sequences that can be amplified using the same primer sequence and detected using the same detection sequence.

The term “mutant-capturing probe”, as used herein, refers to a target-capturing probe that can be used to anneal to a mutant variant of a target gene and selectively make an exonuclease-resistant mutant-specific connection product using the mutant variant as a template. The mutant-capturing probe is preferably a single stranded DNA. The mutant-capturing probe has a target-specific portion (3′-TSS and 5′-TSS), a universal primer recognizing portion (Forward-UPBS and Reverse-UPBS), and a universal detection portion. It can be either a single circularizable polynucleotide or two polynucleotides, each having an exonuclease-resistant end. The key function of mutant-capturing probe is to selectively convert sequence information of a mutant variant into an exonuclease-resistant mutant-specific connection product. The mutant-specific connection product can then be PCR amplified using PCR primers recognizing the universal primer binding site and be detected by a sequence-specific reporter probe recognizing the universal detection site. In some embodiment, the 3′-TSS and 5′-TSS of the mutant-capturing probe comprise sequences hybridize to a region of the target gene that overlaps or is close to a mutated region. In some embodiment, the 3′-TSS and/or 5′-TSS comprises a portion of a mutant sequence and hybridize specifically to a mutant variant. In some embodiment, a blocking probe complementary to the wild-type sequence is used to block formation of an exonuclease-resistant DNA product using the wild-type sequence as a template, allowing selective formation of an exonuclease-resistant mutant-specific connection product.

The term “target-specific sequence (TSS)”, as used herein, refers to a sequence that is complementary to a unique region of a target sequence. The length of a target-specific sequence should be sufficient for the TSS to specifically hybridize to the complementary sequence on the target sequence. It can be, for example, 10 to 40 nucleotides, preferably 15-30 nucleotides, most preferably 18-25 nucleotides. A mutant-capturing probe has a 3′-TSS and a 5′-TSS that are located at the 3′ and 5′ terminal of the probe, respectively. The 3′-TSS and 5′-TSS of the mutant-capturing probe anneal to a region of the target gene that overlaps or is close to a mutated region. In some embodiment, the 3′-TSS and/or the 5′-TSS comprises a sequence complementary to a portion of a mutant sequence which allows selective annealing of the mutant-capturing probe to a mutant variant. For example, The 3′-TSS and/or the 5′-TSS may have a terminal nucleotide matching to a mutated nucleotide. In some embodiment, the 3′-TSS and the 5′-TSS anneal adjacently to a mutated region of a mutant variant. In some embodiment, the 3′-TSS and the 5′-TSS anneal to a target gene to form a nucleic acid duplex with a single nucleotide or a multi-nucleotide gap, wherein the single nucleotide or the multi-nucleotide gap occupies a locus of a mutated region. In some embodiment, a target-capturing probe has a 3′-TSS and a 5′-TSS annealing to a selected region of a wild-type sequence of a target gene. The target-capturing probe is used to detect the presence of a wild-type target sequence. In some embodiment, a target-specific sequence comprises a sequence complementary to a predesigned sequence tag of a target sequence in a sample.

The term “universal primer binding sequences”, as used herein, refers to predesigned sequences that are non-complementary to all the sequences in a nucleic acid sample and are used for PCR primer binding purpose. The PCR primers may bind to the universal primer binding sequence itself or its complement. The PCR primers are designed such that the resulting PCR product encompasses the connected 3′-TSS and 5′-TSS of a target-capturing probe.

The term “breakable site”, as used herein, refers a chemical moiety that is susceptible to photo, enzymatic or chemical cleavage. The breakable site may comprise a RNA sequence, modified nucleotides and/or other non-nucleotide chemical moieties. In some embodiment, the breakable site may incorporate a RNA sequence which can be cleaved by RNAses. In some embodiment, the breakable site comprises uracil nucleotides, which can be converted to a baseless nucleotide by Uracil DNA Glycosylases and be subjected to cleavage by AP endonucleases. In some embodiment, the breakable site comprises modified nucleotides that are susceptible to chemical cleavage. For example, 5-hydroxy-dCTP, 5-hydroxy-dUTP, 7-Deaza-7-nitro-dATP, 7-Deaza-7-nitro-dGTP are modified nucleotides that are subjected to chemical cleavage by KMnO4 and pyrrolidine treatment (Wolf J L, et al. Proc Natl Acad Sci USA., 2002 99(17):11073-8). In some embodiment, the breakable site comprises a photo-cleavable chemical moiety which can be cleaved by exposure to UV light. The photo-cleavable chemical moiety can be, for example, a 9-atom photo-cleavable nucleotide spacer (PC spacer) commercially available from Integrated DNA Technologies (Coralville, Iowa). Photo cleavage of a PC spacer releases an oligo nucleotide with a 5′ phosphate.

The term “exonuclease-resistant end”, as used herein, refers to a chemical moiety or modification incorporated to an end of a nucleotide sequence that renders the nucleotide sequence resistant to exonuclease digestion. The chemical moiety or modification can be modified nucleotide analogues or other chemical structures unrelated to nucleotides. For example, two or more phosphoramidate and phosphorocmonothioate and/or phosphorodithioate linkages can be incorporated at the 5′ or 3′ end of a polynucleotide to render the polynucleotide exonuclease resistant (U.S. Pat. No. 5,256,755). Inverted 3′-3′ or 5′-5′ nucleotide linkage can be incorporated at an end of an polynucleotide that will inhibit degradation by exonucleases. Other modifications such as 2′-Fluoro nucleotides, morpholine oligonucleotides, peptide nucleic acid, and locked nucleic acid can also be incorporated at ends of a polynucleotide to inhibit exonuclease degradation (Iyer A K and He J. Curr Org Synth. 2011, 8(4): 604-614).

The term “universal detection sequence”, as used herein, refers to a predesigned nucleotide sequence that is non-complementary to target genes and can hybridize to a sequence-specific reporter probe to release a detectable signal. “A sequence-specific reporter probe” refers to a reporter probe that comprises a specific nucleotide sequence and a reporter moiety, which can give off a reporting signal upon binding to its complementary sequence. A preferable reporting signal is a fluorescent signal. By coupling to different fluorophores, different sequence-specific reporting probes can be used in the same reaction for multiplexed detection of different target sequences. The universal detection sequence is inserted either between 5′-TSS and Forward-UPBS or between 3′-TSS and Reverse-UPBS. The sequence-specific reporter probe is designed to hybridize to the universal detection sequence itself or the complement thereof on the opposite strand such that a detectable signal is generated only when a new DNA strand encompassing a connected 3′-TSS and 5′-TSS is synthesized during PCR amplification. For example, a universal detection sequence is inserted between the 5′-TSS and the Forward-UPBS, and the sequence that binds to the reporter probe is its complementary sequence on the opposite strand which is synthesized during PCR amplification. The hybridization of the reporter probe to its complementary sequence during PCR amplification will trigger a release of a detectable signal. The same universal detection sequence can be used in different capturing probes for detection of different mutant variants or wild-type target genes. There are many sequence-specific reporter probes that upon binding to a specific sequence, the probes undergo a structural change or are cleaved by an enzyme, resulting in a product that can give off a detectable signal (Marras S A E, et al. Clinica Chimica Acta 2006, 363:48-60). The sequence-specific reporter probes includes, but not limited to, 5′-nuclease Taqman® probes (Holland P M, et al. Proc. Natl Acad. Sci. USA, 1991, 88:7276-7280, and Woo T H S, et al. Anal. Biochem. 1998, 256: 132-134), Scorpion® probes (Thelwell N, et al. Nucl Acids Res 2000, 28(19):3752-3761), and Molecular beacons probes (Tyagi S and Kramer F R Nat. Biotechnol. 1996, 14: 303-308, and Kostrikis L G, et al. Science 1998, 279: 1228-1229), light up probes (Svanvik N, et al. Anal Biochem 2000, 281: 26-35), and adjacent probes (Cardullo R A, et al. Proc Natl Acad Sci USA 1988, 85:8790-4).

The term “a blocking probe”, as used herein, refers to a nucleic acid or modified nucleic acid probe that is complementary to a region of wild-type sequence of a target gene, but is not complementary to the corresponding region of a mutant variant of interest. The duplex formed by the blocking probe and the mutant variant has at least one mismatch, rendering it less stable than the duplex formed by the blocking probe and the wild-type sequence. By choosing the right annealing temperature, the blocking probe will selectively hybridize to the wild-type sequence but not the mutant variant. The modification of nucleic acids that increases the difference in the hybridization strength between perfectly matched and mismatched probe-target duplexes is preferable for this invention, which includes, but not limited to, peptide nucleic acids and locked nucleic acids. A minor groove binder, for example, can be introduced to increase the difference in stability between perfectly complementary and mismatched probe-target hybrids (Kutyavin I V, et al. Nucleic Acids Res. 2000, 28: 655-61). The blocking probe specifically binds to a region in the wild-type sequence that is the counterpart to a mutated region in the mutant variant. The blocking probe can partially overlap with the 3′-TSS and/or the 5′-TSS of mutant-capturing probes. The blocking probe can block the annealing of the TSS to the wild-type sequence, and/or inhibit the extension of 3′-TSS using the wild-type sequence as a template. In some embodiment, the blocking probe is modified to be non-extendable, for example, having a 2′,3′ dideoxynucleotide at the 3′ end or a phosphorylated 3′ end.

The term “target-selective connection”, as used herein, refers to a method for selectively connecting a 3′-TSS and a 5′-TSS of a target-capturing probe to form an exonuclease-resistant target-specific connection product using the target sequence as a template. Both the 3′-TSS and the 5′-TSS anneal to the same strand of the target sequence, and the ends of the 3′-TSS and the 5′-TSS can be connected either by direct ligation or extension/ligation only when the target sequence is present in the sample.

The term “mutant-selective connection”, as used herein, refers to a method for selectively connecting a 3′-TSS and a 5′-TSS of a mutant-capturing probe to form an exonuclease-resistant mutant-specific connection product using a mutant variant as a template. The selective connection is achieved by using a mutant-specific TSS that specifically anneals to mutant variants and/or a wild-type blocking probe that specifically blocks the formation of an exonuclease-resistant product based on the wild-type sequence. In some embodiment, the 3′-TSS and the 5′-TSS anneal adjacently to a mutated region of a mutant variant, and the exonuclease-resistant mutant-specific connection product is formed by direct ligation the 3′-TSS and 5′-TSS. In some embodiment, the 3′-TSS and the 5′-TSS anneal to a target gene to form a DNA duplex with a single nucleotide gap at the locus of a mutated nucleotide. The exonuclease-resistant mutant-specific connection product is formed by single nucleotide extension and ligation. Optionally, a wild-type blocking probe can be used to further increase the selectivity and specificity. In some embodiment, the 3′-TSS and the 5′-TSS anneal to a target gene to form a DNA duplex with a multi-nucleotide gap which overlaps with a mutated region. For mutant variants with known mutations, the 3′-TSS can be designed to incorporate the known mutation at the 3′ end, which allows 3′-TSS to specifically anneal to the mutant variant. The exonuclease-resistant mutant-specific connection product is formed by DNA polymerization and ligation. For mutant variants where the mutated region is known, but the specific mutations are unknown, the 3′-TSS and 5′-TSS are designed to be complementary to the upstream and downstream sequence of the mutated region, respectively. Using a wild-type blocking probe to block extension based on the wild-type sequence, only the 3′-TSS annealed to the mutant variant can be extended by a DNA polymerase and further ligated with the 5′-TSS to form an exonuclease-resistant mutant-specific product.

Using a uniquely designed target-capturing probe, the present invention provides a one tube reaction for easy yet robust detection of target sequences in a nucleic acid sample. The method comprises the following steps:
a. providing a target-capturing probe containing a target-specific portion and a universal primer recognition and universal detection portion;
b. selecting a target-capturing probe from
   i. a single polynucleotide, from 5′ to 3′ end sequentially comprising a 5′-TSS, a Forward-UPBS, a breakable site, a Reverse-UPBS and a 3′-TSS, having a universal detection sequence inserted between the Forward-UPBS and the 5′-TSS or the Reverse-UPBS and the 3′-TSS, or
   ii. two polynucleotides, comprising a first polynucleotide that comprises an exonuclease-resistant 5′end, a Reverse-UPBS, and a 3′-TSS at the 3′ end, and a second polynucleotide that comprises a 5′-TSS at the 5′ end, a Forward-UPBS, and an exonuclease-resistant 3′ end, having a universal detection sequence inserted between the Forward-UPBS and the 5′-TSS or the Reverse-UPBS and the 3′-TSS;
c. contacting the target-capturing probe with the nucleic acid sample under conditions where the 3′-TSS and the 5′-TSS of the target-capturing probe anneal to a complementary target sequence to form a duplex nucleic acid;
d. performing a target-selective connection wherein the 3′-TSS and the 5′-TSS of the target-capturing probe are connected to form a linear or circular exonuclease-resistant target-specific connection product using the target sequence as a template;
e. Optionally, using an exonuclease to digest susceptible nucleic acids, and after the enzymatic digestion, inactivating the exonuclease and/or purifying the exonuclease-resistant target-specific connection products;
f. breaking the circular exonuclease-resistant target-specific connection products, if present, at the breakable site to obtain linear target-specific connection products;
g. amplifying the linear target-specific connection products or the linear exonuclease-resistant target-specific connection products by a polymerase chain reaction using universal PCR primers recognizing the universal primer binding sequences or the complement thereof; and
h. detecting or quantitating the linear target-specific connection products or the linear exonuclease-resistant target-specific connection products as a measurement of the target sequences using a sequence-specific reporter probe recognizing the universal detection sequence.

The invention provides a unique target-capturing probe containing target specific sequences (TSS), universal primer binding sequences (UPBS) and a universal detection sequence. The method starts by adding the target-capturing probe to a denatured nucleic acid sample suspected of containing target sequences of interest under the condition that allows the 3′-TSS and the 5′-TSS of the capturing probes to anneal to the complementary target sequences. If a target sequence is present in the sample, a target-specific connection product will be generated through a target-selective connection of 3′-TSS and 5′-TSS. To increase the amount of target-specific connection product, the denaturing, annealing and target-selective connection steps can be repeated multiple times for linear amplification of the target-specific connection product. The target-capturing probe is designed such that the target-specific connection product is exonuclease resistant. The exonuclease-resistant target-specific connection product is then purified by exonuclease digestion of susceptible nucleic acids including non-connected target-capturing probes and the nucleic acid sequences in the sample. When the target-capturing probe is a circularizable single polynucleotide, the resulting target-specific connection product is in a circular form with a breakable site. The circular target-specific connection product can be linearized by breaking the breakable site. The linearized target-specific connection product can then be PCR amplified using a pair of universal PCR primers and detected or quantified by a sequence-specific reporter probe recognizing the universal detection sequence. The detection and amplification can be combined in a PCR, preferably a quantitative PCR process. The target-specific connection product can also be subjected to for example, sequencing analysis, electrophoresis gel analysis, and mass spectrometry analysis.

One aspect of the invention is to provide a one tube reaction for easy yet robust and sensitive detection of target sequences in a sample. The present invention allows selection, purification, amplification and detection of target sequences in one single tube, which greatly simplifies the operation process and significantly decreases the chances of contamination.

Another aspect of the invention is to separate the target discrimination reaction from the amplification and detection reactions. The invention exploits a target-capturing probe and target-selective connection to convert the representation of target sequences in a sample to target-specific connection products. The target-specific connection products are then amplified and detected using the universal primer binding sequences and the universal detection sequence. This approach greatly simplifies the optimization process for detecting different target sequences, significantly decreases the variation due to difference in primer efficiency, allows detection of multiple target sequences in one reaction, and is easily adaptable to multiplexed and automated assays.

Another aspect of the invention is that it is especially suitable for detecting rare mutants in the presence of excess wild-type sequences. The target-capturing probe can be designed to be a mutant-capturing probe that comprise 3′-TSS and 5′-TSS specific for a mutant variant of interest. The 3′-TSS and 5′-TSS of the mutant-capturing probe can be designed to contain part of the mutated sequences or be in close proximity to the mutated site. The invention uses a blocker probe to block the formation of wild type-specific connection product and uses linear amplification to increase the amount of mutant-specific connection product, further increasing the specificity and selectivity of detecting the mutant variant. Another aspect of the invention is selective amplification and detection of mutant variants with unknown mutations, including fusion mutations with unknown fusion sequences. This is achieved by using a wild-type blocking probe to block the formation of connection products based on wild-type sequences, allowing selective production of connection products based on mutant variants.

Another aspect of the invention is to use a plurality of target-capturing probes for determination of copy number variation of a target gene/chromosome. Using groups of target-capturing probes having TSS regions specific for multiple gene loci on a target or a reference gene/chromosome, the copy number of the target and the reference gene/chromosome can be easily determined, which uses a universal primer sequence shared by all the probes and detection sequences shared for each group of the probes. This method can greatly lower the measurement variation due to the difference in PCR primer efficiency.

### Nucleic Acid Sample

The nucleic acid sample used in the invention can be any preparation of nucleic acids from any sources, in particular, biological sources. In the nucleic acid sample, the nucleic acids suspected of containing the target may be in the form of DNA or RNA. In most cases, RNAs are converted to DNAs using reverse transcription before tested in the invention. The DNA in the sample may be double stranded, single stranded, and double stranded DNA denatured into single stranded DNA. The nucleic acids in the sample may be prepared from any source, including RNA, mRNA, smRNA, rRNA, cDNA, genomic DNA, organellar DNA, synthetic DNA, DNA libraries (e.g. BAC libraries or pooled BAC clones), clone banks or any combinations thereof. The nucleic acids may be DNA sequences with varied lengths, ranging from 20 to hundreds, to thousands, or even millions of nucleotides. The invention focuses on retrieving information from the relatively small mutated regions of target genes. It can work equally well with short as well as long DNA sequences. This is especially advantageous when working with DNA preparations from sources such as circulating cell free DNA and Formalin-fixed, paraffin-embedded (FFPE) tissue DNA, where only short DNA fragments can be obtained due to DNA degradation. The nucleic acids may contain artificial sequence tags added to one or both ends. These sequence tags are unique predesigned sequences that are non-complementary to original sequences in the sample. Mutant-capturing probes having TTS complementary to the sequence tags can be used to capture mutant variants with unknown sequences.

### Target/Mutant-Capturing Probes

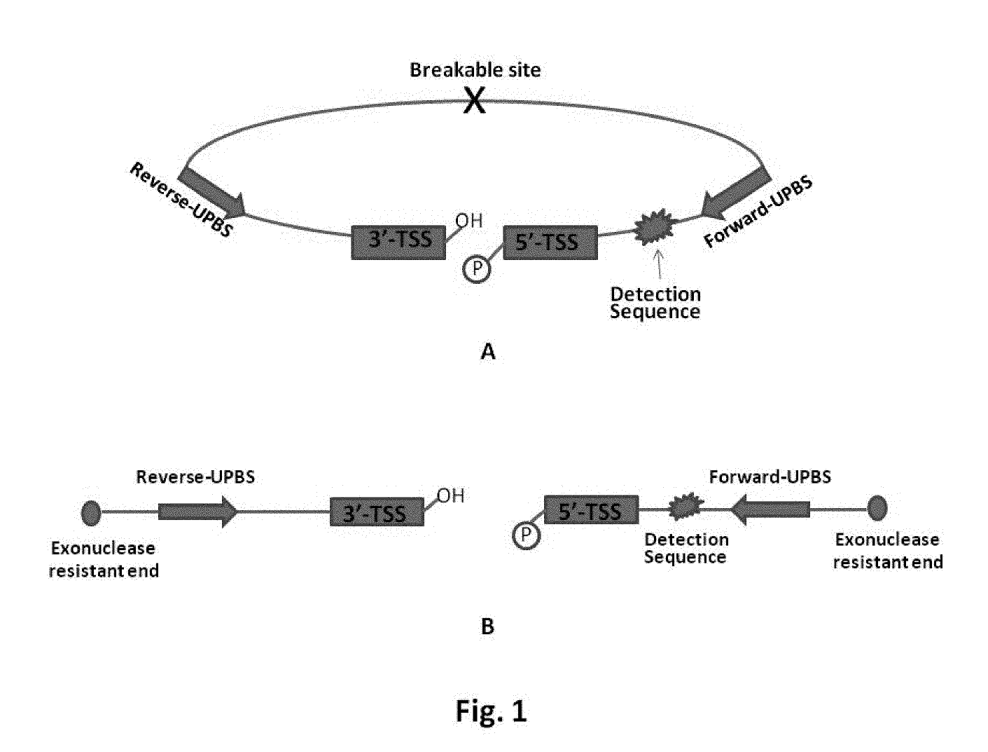

An important feature of the invention resides in the unique design of a target-capturing probe that is used to retrieve the representation information of target sequences in a nucleic acid sample and convert this information into a target-specific connection product, which can be amplified using a pair of universal primers and detected by a universal reporter probe. The target-capturing probe has a pair of target-specific sections (3′-TSS and 5′-TSS), a pair of universal primer recognizing sections (Forward-UPBS and Reverse-UPBS), and a universal detection section. In some embodiment, the target-capturing probe comprises a circularizable polypeptide from 5′ to 3′ end sequentially comprising a 5′-TSS, Forward-UPBS, a breakable site, Reverse-UPBS and a 3′-TSS, having a universal detection sequence inserted between the Forward-UPBS and the 5′-TSS or the Reverse-UPBS and the 3′-TSS (FIG. 1A). This circularizable target-capturing probe can anneal to a target sequence and produce a circular exonuclease-resistant target-specific connection product by connecting the 3′-TSS and 5′-TSS. This circular target-specific connection product can be linearized by breaking the breakable site before subjecting it to PCR amplification and detection. In some embodiment, the target-capturing probe comprises two polynucleotides that comprises a first polynucleotide containing an exonuclease-resistant 5′ end, a Reverse-UPBS, and a 3′-TSS at the 3′ end, and a second polynucleotide containing a 5′-TSS at the 5′ end, a Forward-UPBS, and an exonuclease-resistant 3′ end, having a universal detection sequence inserted between the Forward-UPBS and the 5′-TSS or the Reverse-UPBS and the 3′-TSS (FIG. 1B). This pair of target-capturing probe can anneal to a target sequence and produce a linear exonuclease-resistant target-specific connection product by connecting the 3′-TSS and the 5′-TSS.

The 3′-TSS and the 5′-TSS are designed to be complementary to a first and a second region, respectively, on the same strand of a target sequence. The first and the second region of the target sequence may be adjacent to each other or be separated from each other by one or more nucleotides. The length of the TSS varies from 10 to 40 nucleotides, preferably 15 to 25 nucleotides, more preferably 18 to 22 nucleotides. The 3′-TSS has a free 3′ hydroxyl end and the 5′-TSS has a free 5′ phosphorylated end, allowing them to be connected by direct ligation when annealed adjacently on the target sequence. Or the 3′-TSS can be extended by DNA polymerization and the extended 3′-TSS can be ligated to 5′-TSS.

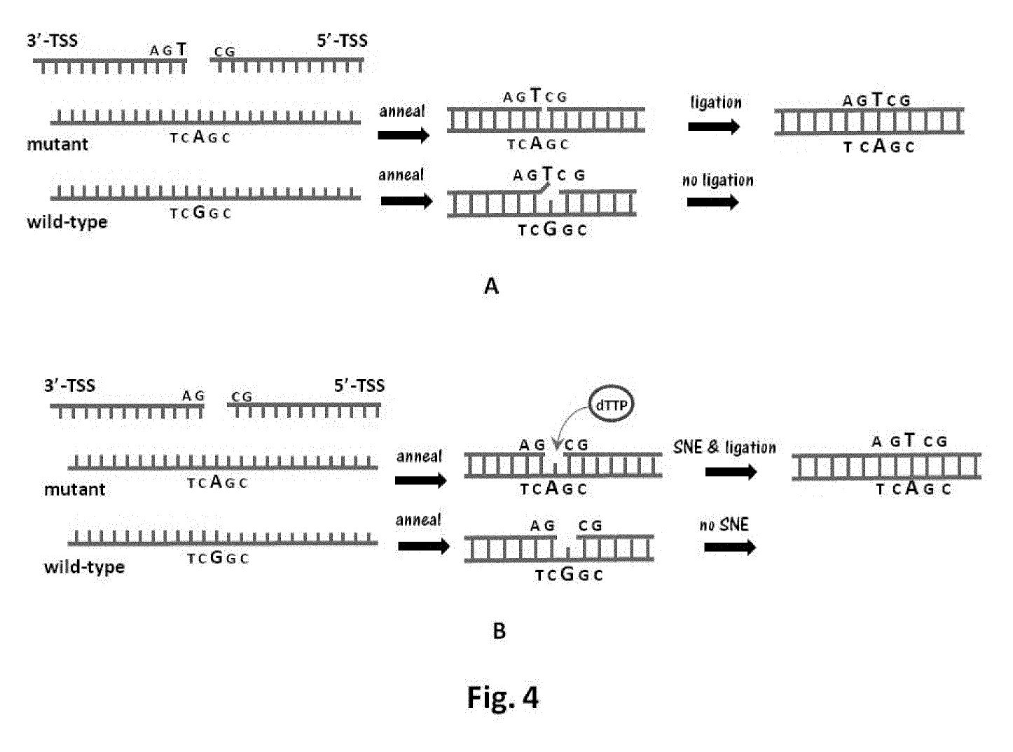

In some embodiment, the target sequence is a mutant variant of a target gene, and the target-capturing probe is thus a mutant-capturing probe. In some embodiment, It is designed that the 3′-TSS and/or the 5′-TSS of the mutant-capturing probe comprise terminal mutated nucleotide and they anneal adjacently to a mutated region of the mutant variant, allowing direct ligation to connect the 3′-TSS and the 5′-TSS to form a mutant-specific connection product (FIG. 4A). When the 3′-TSS and the 5′-TSS anneal to a wild-type sequence, the ligation does not occur because of mismatched end nucleotides. To further increase the specificity, a blocking probe is used to prevent the 3′-TSS and 5′-TSS from annealing to the wild-sequence.

In some embodiment, the 3′-TSS and the 5′-TSS of a mutant-capturing probe are designed to anneal to the target sequence to form a DNA duplex with one single nucleotide gap at the locus of a mutated nucleotide (FIG. 4B). After the annealing, perform single nucleotide extension and ligation in the presence of only one type of nucleotide that matches to the mutated nucleotide, which generates a mutant-specific connection product. In some embodiment, there are multiple mutant variants having different nucleotides at a single mutation locus. It is desirable to detect whether there is a mutation regardless of the identity of the mutation. The single nucleotide extension and subsequent ligation can be performed in the presence of three types of nucleotide where the fourth nucleotide is the one matching to the wild-type genotype. In addition, a blocking probe can be added to block the annealing of the 3′-TSS and the 5′-TSS to the wild-type sequence.

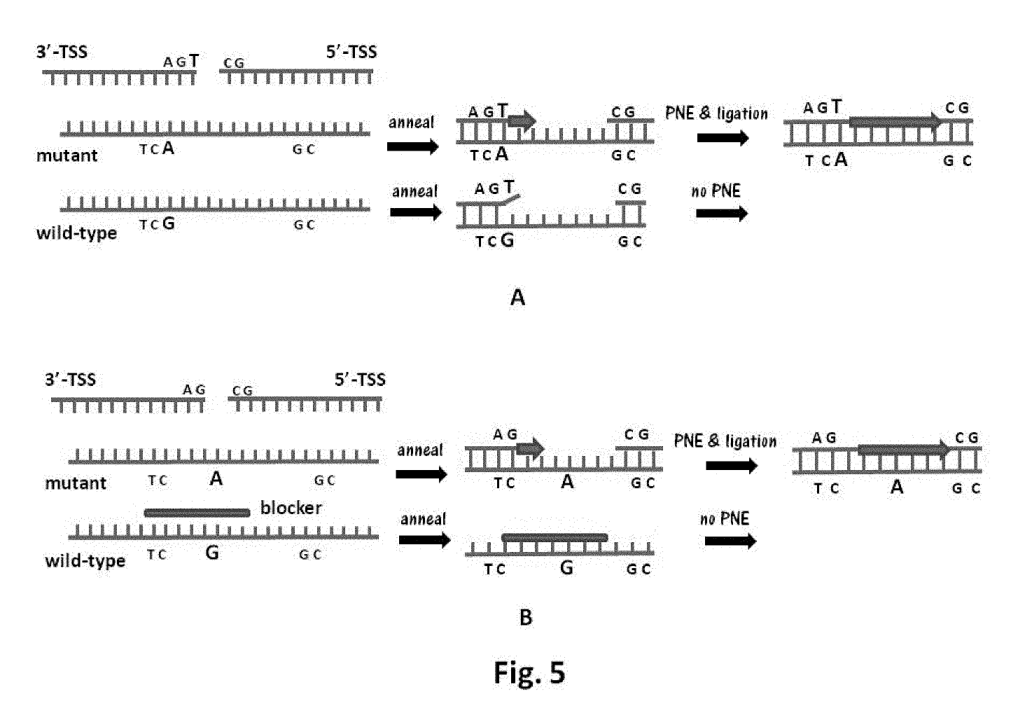

In some embodiment, the 3′-TSS and the 5′-TSS of a mutant-capturing probe are designed to anneal to the target sequence to form a DNA duplex with a multi-nucleotide gap at the locus of a mutated region (FIG. 5A, 5B). The mutant-specific connection product is formed by extending the 3′-TSS by DNA polymerization using a DNA polymerase until it is adjacent to the 5′-TSS, and then ligating the extended 3′-TSS and 5′-TSS. It is preferable that the DNA polymerase used here does not have 5′->3′ exonuclease activity and does not express strand displacement activity. Examples of such enzymes are Klenow fragment of DNA polymerase I, T4 DNA polymerase, and T7 DNA polymerase. If the mutant sequence is known, the 3′-TSS and optionally the 5′-TSS may comprise a portion of the mutant sequences, particularly, the 3′-TSS may have a mutated nucleotide at its 3′ end. This allows the 3′-TSS and the 5′-TSS to preferably anneal to the mutant variant compared to the wild-type sequence. Additionally, a blocking probe for wild-type sequence can be added for increased specificity. If the mutant sequence is unknown but the locus of mutation is known, the 3′-TSS and the 5′-TSS are designed to be flanking the mutated region. A blocking probe complementary to the wild-type counterpart sequence of the mutated locus can be used to select mutant variants. The blocking probe will form a perfectly matched duplex with the wild-type sequence, while the duplex formed between the blocking probe and the mutant variant will have at least one mismatch. The melting temperature Tₘ of the blocking probe-wild type duplex will be higher than Tₘ of the blocking probe-mutant variant duplex. By selecting an annealing temperature between the two melting temperature, the blocking probe will selectively block the extension using the wild-type sequence as a template. Depending on the difference of the two melting temperatures, the selective blocking may not be complete and false positive may be likely. One way to determine whether the detected signal is from mutant variants or partial blockage of wild-type sequence is to compare the detected signal to that of a wild-type only control. Alternatively, the amplified TSS connection products can be subjected to sequencing analysis to determine the presence of mutant variants.

In some embodiment, the mutant variant is a gene fusion mutation where the fusion location of a first fusion gene is known but the second fusion gene sequence is not known. The present invention can be used to detect the presence of such gene fusion mutants with unknown sequences. First, ligate a 5′ sequence tag to nucleic acids suspected to contain gene fusion mutants. The 3′-TSS is designed to be complementary to a region of the first fusion gene upstream of the fusion location. The 5′-TSS is designed to be complementary to the sequence tag. A blocking probe is designed to be complementary to a region of the wild-type sequence across the fusion location, which will specifically hybridize to the wild-type sequence across the fusion location, but not the gene fusion mutant. The mutant-capturing probe anneals to the fusion mutant variant and is extended from the 3′-TSS by DNA polymerization until the extended strand meets the 5′-TSS, which can be ligated together to form a connection product specific for the fusion mutant. On the other hand, the blocking probe blocks the annealing of the 3′-TSS to the wild-type sequence and/or extension of 3′-TSS using wild-type sequence as a template, resulting in no formation of connection products templated on the wild-type sequence. The length of the blocking probe can have at least 20 to 40, 40 to 100, 100 to 200, 200 to 400 nucleotides and more, preferably 40 to 150 nucleotides. The blocking probe needs to encompass the fusion junction locus, preferably in the middle of the blocking probe sequence. As long as the fusion junction is covered by the blocking probe, the exact locus of the fusion junction is not required to be known. Using the blocking probe will allow 3′-TSS extension only using gene fusion mutant variants as templates. This method can be used to obtain sequence information from all the gene fusion mutants having this particular gene fusion location.

In some embodiment, the target-capturing probe is used to detect a wild-type sequence of a target gene. The target-capturing probes can be used to detect multiple target sequences in a single-tube reaction. A plurality of target-capturing probes comprising different target-specific sequences with the same universal primer binding sequences and the same universal detection sequence can be used to simultaneously detect a plurality of target sequences. In some embodiment, the plurality of target-capturing probes can be divided into different groups, each having a distinct detection sequence but sharing the same primer binding sequences. In some embodiment, the method is used to detect copy number variation of a target gene/chromosome. A plurality of target-capturing probes comprising a first and a second group of target-capturing probes are added to the nucleic acid sample. Target-capturing probe of the first group have 3′-TSS and 5′-TSS complementary to sequences of different gene loci on the target gene/chromosome and all the target-capturing probes of the first group share a first universal detection sequence. Target-capturing probes of the second group has 3′-TSS and 5′-TSS complementary to sequences of different gene loci on the reference gene/chromosome and all the target-capturing probes of the second group share a second universal detection sequence. All the target-capturing probes are preferably having the same primer binding sequences. Using the same primer binding sequences for all the target-capturing probes can minimize the amplification variation due to difference in PCR efficiency of different PCR primers. Preferably, the gene loci selected for analysis are evenly distributed on the target and reference gene, and are non-overlapping to each other. The number of selected gene loci on the target and reference gene/chromosome is the same. The first and the second detection sequence are recognized by different sequence-specific reporter probes that are coupled with different fluorophores. This allows measurement of the amount of the target and reference gene/chromosome in the same tube using different fluorophores for detection. Preferably, the copy numbers of the multiple gene loci on the target gene/chromosome and the reference gene/chromosome are determined using a digital PCR to detect fluorescence emitted by respective sequence-specific reporter probes of the first and the second detection sequence. The ratio of the copy number of the target gene/chromosome vs. the reference gene/chromosome is used to determine the presence of a copy number variation.

In some embodiment, this method can be used to detect the copy number variation of the same gene in different samples, for example, a patient sample vs. a normal sample. In this case, the target-capturing probes have the same target-specific portions and different detection sequences are used for different samples. Once the target specific connection products for different samples are separately captured using target-capturing probes with different detection sequences, they are pooled together and the gene copy number of different samples are detected by the respective sequence-specific reporter probes.

### Blocking Probe

A blocking probe comprises a nucleotide sequence that is fully complementary to a region of a wild-type sequence, but not to the corresponding region in mutant variants. The blocking probe forms a perfectly matched duplex with the wild-type sequence while the blocking probe-mutant duplex has at least one mismatch. The melting temperature of a perfectly matched duplex (Tₘ₁) is usually higher than that of a duplex with mismatches (Tₘ₂). By choosing an annealing temperature between Tₘ₁ and Tₘ₂, it may be possible to find a annealing temperature that most wild-type sequence are hybridized with the blocking probe while most of the mutant sequences are unoccupied. It is therefore desirable to find nucleotide modification that leads to bigger difference in stability between a perfectly matched and mismatched duplex. One example is peptide nucleic acid probes (PNA). PNAs are DNA mimics in which the deoxyribose-phosphate backbone is replaced by N-(2-aminoethyl)-glycine units linked by peptide bonds. The PNA can form a duplex with a complementary DNA. A single base mismatch in a PNA-DNA duplex is much more destabilizing than in the corresponding DNA-DNA duplex, making it a good candidate to be used in a blocking probe. Another example is locked nucleic acid probes (LNA). LNA is a modified RNA where the 2′ oxygen and 4′ carbon of the ribose moiety is connected by an extra bridge. LNA nucleotides can make base pairing with normal nucleotides and can be mixed with DNA and RNA residues. Blocking probes made of LNA nucleotides can have increased specificity and sensitivity. The blocking probes can also be linked to minor groove binder to increase the specificity of the probe and ability to discriminating mismatched nucleotides. It is also desirable to include modification that makes the blocking probe resistant to exonuclease and non-extendable. For example, adding two or more phosphoramidate and phosphorocmonothioate and/or phosphorodithioate linkages at the 5′ or 3′ end of a polynucleotide can render the polynucleotide to be exonuclease resistant. Incorporating a 2′,3′ dideoxynucleotide to the 3′ end or adding a 3′ end phosphorylated can make the blocking probe non-extendable.

### Target/Mutant-Selective Connection

A key feature of the present invention is to use a target-capturing probe to perform a target-selective connection to capture the representation information of target sequences into target-specific connection products. The target-capturing probe comprises a 3′ and 5′ target-specific sequence that first anneal to the same strand of a target sequence in the sample, which is then connected by direct ligation or extension/ligation to form a target-specific connection product. The target-specific connection product can only be generated when the target sequence is present in the sample. Each copy of the target-specific connection product is a representation of the target sequence in the sample. The target-capturing probe can be used to capture a wild-type or a mutant variant of a target gene.

In a preferred embodiment, a mutant-capturing probe is used for capturing a mutant variant via a mutant-selective connection, which is achieved by using mutant-specific sequence to capture mutant variants and/or using a wild-type blocking probe to block formation of connection products based on wild-type sequences.

The first type of mutant-selective connection is mutant-specific ligation which is applicable to mutants with known mutations. The 3′-TSS or the 5′-TSS of the mutant-capturing probe is designed to be exactly matched to mutated nucleotides, preferably, at least one mutated nucleotide is located at 3′ end of the 3′-TSS and/or 5′end of the 5′-TSS. The 3′-TSS and the 5′-TSS are designed to anneal adjacently to the mutated region of the mutant variant. Mix the mutant-capturing probe with a nucleic acid sample, perform a cycle of denaturing, annealing and ligation to generate a mutant-specific connection product. When the mutant variant is present in the nucleic acid sample, the 3′-TSS and 5′-TSS will anneal adjacently to the mutated region and be ligated together using a thermostable DNA ligase to form the mutant-specific connection product. If needed, the cycle of denaturing, annealing and ligation can be repeated multiple times to achieve linear amplification of the mutant-specific connection product. The annealing temperature is chosen such that the mismatched hybridization between the TSS and the wild-type sequence is unstable. Additionally, a blocking probe fully complementary to the wild-type sequence can be added to block the annealing of the mutant-capturing probe to the wild-type sequence, thus further increasing annealing specificity. Due to the sequence specificity of DNA ligase, little or no ligation will occur on mismatched duplexes of the probe and wild-type sequence. To increase the ligation specificity, the ligation can be performed at the elevated ligation temperature (e.g. 50-65° C.). Examples of thermostable ligase includes “Ligase 65” from MRC Holland, Amsterdam and Taq ligase from New England Biolabs (Ipswich, Mass.). This method exploits the sequence specificity of DNA ligase and specific blockage by the blocking probe to select for mutant variants, offering quite high selectivity and specificity.

The second type of mutant-selective connection combines single nucleotide extension with ligation which is applicable to mutants with known or unknown mutations. The 3′-TSS and the 5′-TSS are designed to anneal to the mutated region of the mutant variant with one nucleotide gap, wherein the gap is at the locus of a mutated nucleotide. The identity of the mutated nucleotide is not required to be known. In one embodiment, the mutant-capturing probe is annealed to a target sequence, and only one of the four types of nucleotides is provided together with DNA polymerase and DNA ligase. The single nucleotide extension and subsequent ligation can occur only when a matched nucleotide is provided. The suitable DNA polymerase should possess 5′->3′ DNA polymerase activity without 5′->3′ exonuclease activity, and does not express strand displacement activity. In some embodiment, the identity of the mutated nucleotide is not unknown or it is desirable to determine if any mutant variant is present regardless of the identity of the mutated nucleotide. The above mutant-capturing probe is annealed to a target sequence in the presence with a DNA polymerase, all the four types nucleotides, a DNA ligase, and a wild-type blocking probe. Because the wild-type sequence is hybridized to the blocking probe and is unavailable to anneal to the mutant-capturing probe, only mutant variants can anneal to the mutant-capturing probe and direct the generation of mutant-specific connection products. To increase specificity, only three of the four types of nucleotides will be provided in the reaction, wherein the nucleotide matching to the wild-type genotype is not included in the reaction. This method exploits the sequence specificity of DNA polymerase and DNA ligase, and specific blockage by the blocking probe to select for mutant variants, offering very high selectivity and specificity.

The third type of mutant-selective connection involves DNA polymerization of multiple nucleotides and ligation, which can be used to detect mutants with known or unknown mutations. The 3′-TSS and the 5′-TSS are designed to anneal to the mutated region of the mutant variant with a multi-nucleotide gap, wherein the gap encompasses a mutated region. The sequence of the mutated region is not required to be known. In one embodiment, the sequence of the mutated region is known. The 3′-TSS can be designed to comprise a sequence matching to a portion of the mutated region with one mutated nucleotide at the 3′ end. The 5′-TSS may be designed to be complementary to a sequence downstream of the mutated region, or can partially overlap with the mutated region. This mutant-capturing probe will specifically anneal to the mutant variant and use the mutant variant as the template to make a mutant-specific connection product. The DNA polymerase suitable for this application should have no 3′->5′ or 5′->3′ exonuclease activity and does not express strand displacement activity, such as Sulfolobus DNA Polymerase IV from New England Labs. Additionally, a blocking probe can be used to block the formation of connection product based on the wild-type sequence.

In some embodiment, the sequence of the mutated region is unknown. The 3′-TSS and the 5′-TSS are designed to anneal to target sequences that are upstream and downstream of the mutated region, respectively. A non-extendable blocking probe that is fully complementary to the wild-type counterpart sequence of the putative mutated region is used to selectively block the annealing of the 3′-TSS to the wild-type sequence and/or the extension of the 3′-TSS based on the wild-type sequence, allowing selective capture of mutant sequence information. The blocking probe may comprise a sequence partially overlapping with the 3′-TSS. In some embodiment, the mutant is a fusion gene where the downstream fusion gene sequence is unknown. The mutant has a first upstream fusion gene and a second downstream fusion gene, wherein the fusion locus is known but the sequence of the second downstream fusion gene is not known. A sequence tag should be added to the ends of the nucleic acids in the nucleic acid sample. Methods for ligating a sequence tag to one end or both ends of nucleic acids are known in the art (US Patent Application Nos. 20160304948 and 20120156729; Head S R, et al. Biotechniques. 2015, 56(2): 61-passim). The 3′-TSS can be designed to be complementary to a region in the first fusion gene upstream of the gene fusion locus. The 5′-TSS is designed to be complementary to the 5′ sequence tag of the target gene. A non-extendable blocking probe is designed to be complementary to a region of the wild-type sequence across the gene fusion junction. Perform DNA polymerization using 3′-TSS as the extension primer in the presence of the non-extendable blocking probe. Since annealing of 3′-TSS to the wild-type sequence is blocked by the non-extendable blocking probe, 3′-TSS preferably anneals to the gene fusion mutant variants and makes extension and connection products of the fusion mutants. This method can be used to detect all the different gene fusion mutants having this particular gene fusion locus.

### Exonuclease Treatment

The target-selective connection produces exonuclease-resistant target-specific connection products, which can be a circular product or a linear product with two exonuclease-resistant ends. Exonuclease treatment is used to remove non-connected target-capturing probes and nucleic acids in the sample. Preferable, the exonuclease used here should has 3′->5′ and 5′->3′ exonuclease activity and is able to digest ssDNA and dsDNA. The exonuclease may be a single exonuclease or a mixture of multiple exonucleases. After enzyme digestion, the exonuclease should be inactivated by heating (e.g. 95° C. for 20 minutes). If the target-specific connection product is circular, it can be linearized by breaking the breakable site. There are many methods to incorporate a breakable site that is susceptible to photo, enzymatic or chemical cleavage as described herein elsewhere. One preferable method is to incorporate multiple dU into the breakable site, which can be which can be converted to a baseless nucleotide by Uracil DNA Glycosylases and be subjected to cleavage by AP endonucleases.

### Amplification and Detection

The target-capturing probe is equipped with universal primer binding sequence and universal detection sequence that can be used in different probes for amplification and detection of different target sequences. Using universal primers for PCR amplification of different target genes can greatly reduce the variation due to the difference in PCR efficiency among different PCR primers. It simplifies the optimization process for multiplexed PCR of different targets and makes it easily adaptable to automated operation. While it is common to use target sequence-specific detection probes, using a universal sequence-specific detection probe for detecting different target sequences is a unique feature of the invention. Using a universal detection probe can maximize the detection efficiency regardless of differences in target sequences. Using a universal detection probe allows detection of tens, hundreds, even thousands of different target sequences in one single tube. This is especially useful for detecting whether one of many disease-related mutations is present in a patient's sample or measuring hundreds or thousands of gene loci on a chromosome for detection of copy number variations.

The sequence-specific reporter probes comprise a target recognizing sequence that allows them to detect the complementary sequence in a sample. The sequence-specific reporter probes give off a detectable signal (e.g. a fluorescent signal) upon hybridization to its complementary sequence. It is desirable to have reporter probes that hybridize to the detection sequence or the complement thereof during the PCR process but don't interfere the process of PCR. One example is 5′-nuclease Taqman® probe which comprises an oligonucleotide probe with a fluorophore attached to the 5′-end and a quencher at the 3′-end. Without activation, the 3′-end quencher will quench the fluorescence of 5′-end fluorophore. The Taqman® probes anneal to the detection sequence or the complement thereof during the primer extension. When a DNA polymerase meets the 5′ end of the Taqman® probe, the 5′ to 3′ exonuclease activity of the DNA polymerase degrades the probe and releases the 5′ fluorophore from it, allowing fluorescence of the fluorophore. To detect a DNA strand having a connected 3′-TSS and 5′-TSS or complement thereof, the reporter probe binding sequence should be located at the downstream of the connected TSS. For example, it can be located between the 3′-TSS and the Reverse-UPBS on the target-specific connection product, or be located between the complement of Forward-UPBS and the 5′-TSS on the opposite strand. Choosing a reporter probe binding sequence on the opposite strand is preferable since the opposite strand can only be generated during the PCR amplification. When choosing a reporter binding sequence on the strand of the mutant-specific connection product, it is possible that the left-over un-connected mutant-specific probes may generate false-positive signals and increase the background noise. Another example is molecular beacon probe, a hairpin shaped oligonucleotide with an internally quenched fluorophore. When molecular beacon probes bind to their complementary sequence, the quencher and the fluorophore is separated and the fluorescence is restored. The molecular beacon probes anneal to the detection sequence at the annealing temperature and dissociate from the detection sequence at the extension temperature. They can be used for real time monitoring of the production of the detection sequences. It is preferred that the molecular beacon probe binding sequence is located on the opposite stand of the original mutant-specific connection product strand. These sequence-specific reporter probes can be used in quantitative PCR or digital PCR for detecting the real time production of PCR products containing the universal detection sequence or the complement thereof.

### Preparation of Capturing Probes

The capturing probes used in the invention including mutant- and target-capturing probes have two formats. The first format has two polypeptides, each having a length around 40-70 nucleotides, which can be chemically synthesized easily. The second format has one polynucleotide with a length of more than 100 nucleotides. The yield and efficiency of chemical synthesis becomes very low for polynucleotides with more than 100 nucleotides. Disclosed herein is a method to make a single polynucleotide capturing probe with more than 100 nucleotides. Another aspect of this method is to incorporate multiple dUracil nucleotides to the breakable site of capturing probes.

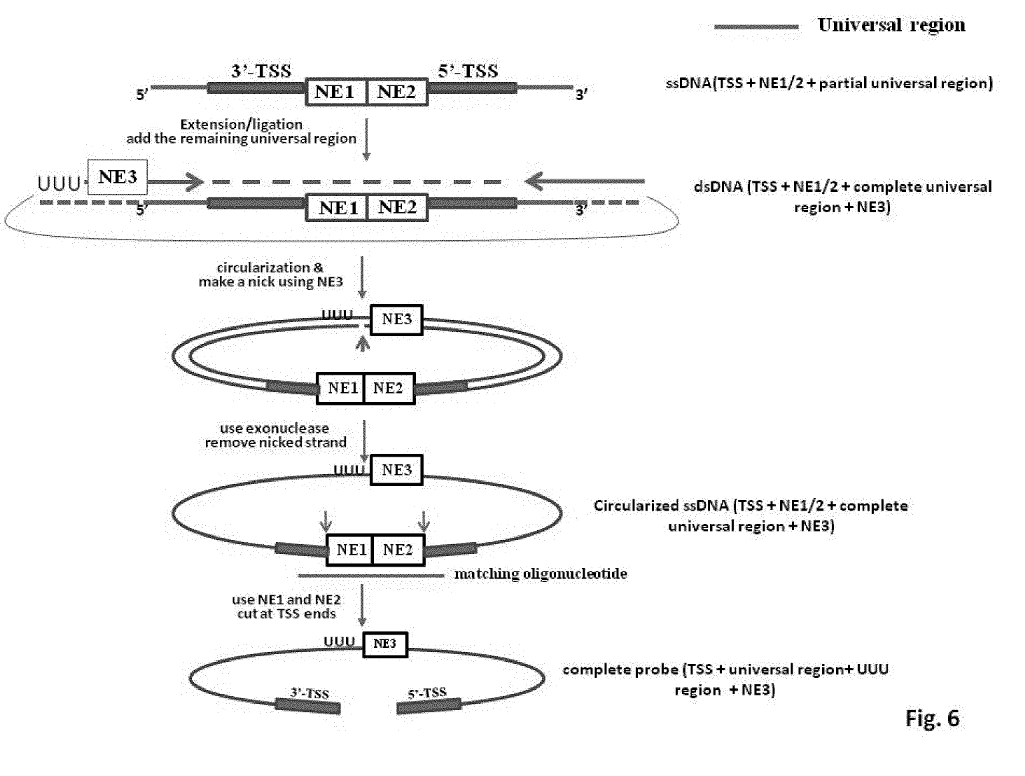

The capturing probe has a target-specific portion (3′-TSS and 5′-TSS) that is specific for each probe, and a universal portion that can be shared by different probes, including a universal primer binding sequence (Forward-UPBS and Reverse-UPBS), a universal detection sequence, and a breakable site. The strategy is to first chemically synthesize the 3′-TSS and 5′-TSS in a head-to-head orientation and part of universal portion of the probes, wherein the total length of the synthesized polynucleotide is no more than 100 nucleotides. Two nicking endonuclease restriction sites (NE1-NE2 site) are introduced between 3′-TSS and 5′-TSS such that digestion by the nicking endonucleases of the double stranded DNA will generate two nicks exactly at the 3′ end of the 3′-TSS and the 5′end of the 5′-TSS on the instant strand, namely the retrieving strand (see FIG. 6). The nicking endonuclease combination suitable for the invention is, for example, Nt. BsmA1 and Nb BsrDI. A synthesized polynucleotide may comprise, for example, from 5′ to 3′, Reverse-UPBS (20 nt), 3′-TSS (20 nt), NE1-NE2 site (12 nt), 5′-TSS (20 nt) and universal detection sequence (20 nt).

Secondly, perform PCR amplification to add the remaining part of universal portion including the multiple dU region or an endonuclease restriction site as the breakable site. For example, the forward primer comprises 5′-(Forward-UPBS, the universal detection sequence)-3′ and the reverse primer comprises 5′-(3×dU, NE3 site, Reverse-UPBS)-3′. The NE3 is a third nicking enzyme restriction site that is different from the NE1 and NE2 sites, and the NE3 nicking enzyme can make a nick on the non-retrieving strand without dU.

The PCR product are circularized by a blunt end ligation. The NE3 nicking enzyme is used to make a nick on the non-retrieving strand and an exonuclease is used to digest the non-retrieving strand, leaving a single-stranded circular retrieving strand with dU breakable site. Alternatively, an endonuclease restriction site can be used in the place of dU as the breakable site.

Synthesize a matching oligonucleotide having a center region complementary to the NE1-NE2 site on the retrieving strand and random nucleotide sequences at both ends. The length of the random nucleotide sequence can be 2 to 10, preferably 4-6 nucleotides. Anneal the matching oligonucleotide to the NE1-NE2 site on the retrieving strand and use NE1 and NE2 nicking enzymes to cut two nicks at the exact end of the 3′-TSS and the 5′-TSS, and generate a single-stranded capturing probe with a complete universal region including a dU breakable site in the middle and the 3′-TSS and the 5′-TSS at the both ends.

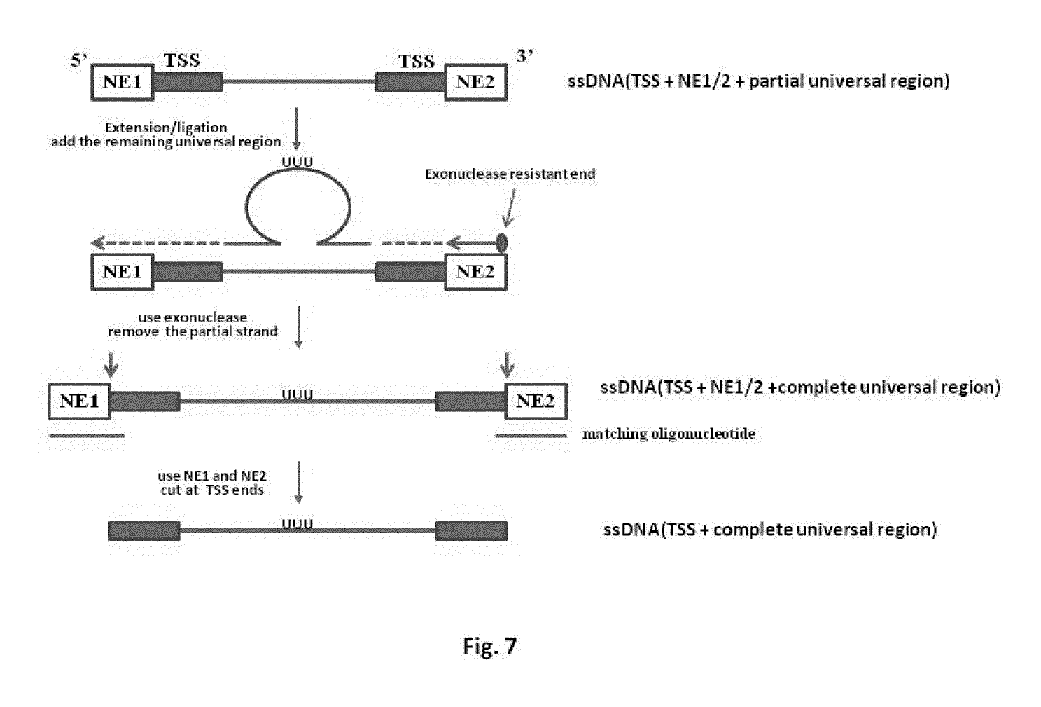

Another method to add a nucleotide sequence to a chemically synthesized probe uses an extension-ligation strategy (see FIG. 7). A complete capturing probe includes a target-specific portion (˜40 nucleotides) and a universal portion (˜70 nucleotides). With nicking enzyme restriction sites (10-12 nucleotides) added to both ends, the final polynucleotide product has a length of 130-134 nucleotides. Synthesize a partial probe with less than 100 nucleotides comprising nicking enzyme restriction sites, 3′-TSS, 5′-TSS, and a partial universal sequence. Anneal a complete universal sequence to the partial universal sequence of the partial probe. Extend the 3′ of the complete universal sequence using the partial probe as a template to fill the 3′ end. Use a sequence complementary to the 3′ nicking enzyme restriction site as a 5′ primer and extend it until it reaches the 5′ end of the complete universal sequence, which can be ligated to form a complete probe having nicking enzyme restriction sites, target-specific sequences and a complete universal sequence (see FIG. 6). The 5′ primer is incorporated with an exonuclease resistant end so as to make the resulting complete probe resistant to exonuclease treatment. After the extension/ligation reaction, the partial probe is removed by an exonuclease treatment while the complete probe is resistant to the exonuclease treatment. A pair of matching oligonucleotides are added to the complete probe with nicking enzyme restriction sites at both ends. Use the two restriction enzyme to cut at the exact ends of the target-specific sequences, thus generating a complete capturing probe with target-specific sequences at both ends and the universal sequences in the middle.

### EXAMPLES

The following examples are intended to further illustrate but not limit the scope of the invention.

### Example 1. Simultaneous Detection of EGFR Mutants

The epidermal growth factor receptor (EGFR) is a receptor tyrosine kinase that has many cancer-related mutations. Patients bearing so called activating mutations of EGFR are sensitive to EGFR tyrosine kinase inhibitor (TKI) drugs such as gefitinib and erlotinib. The TKI-sensitive EGFR mutations include L858R, L861Q, G719S, T783A, etc. Another group of EGFR mutations make patients bearing those mutation develop TKI drug resistance, including T790M, L747S, D761Y, V769M, etc. The present invention can be used to detect whether a patient carries a sensitive or resistant EGFR mutation in one single reaction.

### Mutant-Capturing Probe Design

Two groups of mutant-capturing probes are designed for above-mentioned eight EGFR mutations. The first group for detection of sensitive mutants has four mutant-capturing probes each having a pair of target-specific sequences that are complementary to the mutated region of L858R, L861Q, G719S or T783A mutant. The second group for detection of resistant mutants has four mutant-capturing probes each having a pair of target-specific sequences that are complementary to the mutated region of T790M, L747S, D761Y or V769M mutant. The 3′-TSS and 5′-TSS of each probe are designed to anneal adjacently to the mutated region with the 3′-TSS having a 3′ terminal nucleotide matching to the mutated nucleotide. Probes of the first and the second group share the same universal primer binding sequence, but each group has a distinct universal detection sequence shared within the group. Pool the mutant-capturing probes of both groups to make a probe mixture. For each mutated region, make a wild-type blocking probe of 60 nucleotides long that is complementary to a wild-type region spanning the mutated region. The blocking probe can be linked to a minor groove binder to increase its specificity.

### Generation of Mutant-Specific Connection Products

Circulating DNA is prepared from a patient's blood sample and fragmented into 200-bp fragments. Mix the pooled mutant-capturing probes, the pooled blocking probes, the circulating DNA sample, a thermostable Taq DNA ligase in an appropriate reaction buffer. Perform 10 cycles of denaturing, annealing and ligation to generate mutant-specific connection products if any of the eight mutations is present in the sample. The original amount of mutant DNA is amplified ten times after 10 reaction cycles. The reaction mixture is then subjected to exonuclease digestion to degrade non-connected capturing probes and DNA sequences from the sample. The DNA ligase and exonuclease can be inactivated at 95° C. for 30 minutes. After inactivation of the exonuclease, genomic DNA sample comprising EGFR wild-type sequence is added to the reaction mixture to be used as a reference DNA.

### Amplification and Detection

Add universal PCR primers, three sequence-specific Taqman® probes, primers for amplification of reference DNA, Taq DNA polymerase, dNTPs and suitable buffering components to the reaction mixture. The three sequence-specific Taqman® probes have different fluorophores, including two probes for detecting the universal detection sequence of the two EGFR mutant groups, and one probe for detecting the reference DNA. Perform a qPCR and detect the emitted fluorescence to determine if the patient carries at least a drug-sensitive mutant, a drug-resistant mutant or both, depending on the type of matching fluorescence detected.

### Example 2. Detection of Copy Number Variation in HER2 Gene

The invented method is especially suitable for calculating copy number variation of a gene or chromosome by measuring the copy number of multiple loci on a target gene/chromosome in comparison to a reference gene/chromosome. This example use the invented method to determine a copy number variation of HER2 gene in a cancer patient. The reference gene used is RNase P, which is known to have the normal copy number.

### Design of Target-Capturing Probes

Chemically synthesize 50 target-capturing probes for HER2 gene and RNase P gene, respectively. The target-specific sequences for each capturing probe are equally distributed on HER2 or RNase P gene without any overlap. Each 3′-TSS and 5′-TSS has 20 nucleotides and are designed to anneal to the target gene with a 5-nucleotide gap. All the capturing probes share the same universal PCR primer binding sequences. The capturing probes targeting to HER2 and RNase P contain different universal detection sequences coupled to a FAM and a VIC fluorophore, respectively. All the 100 capturing probes are pooled together to make a probe mixture.

### Generation of Target-Specific Connection Products

Mix a genomic DNA sample with the pooled target-capturing probes, a thermostable Taq DNA ligase, a DNA polymerase, dNTPs in an appropriate reaction buffer. Perform one cycle of denaturing, annealing, DNA polymerization and ligation to generate target-specific connection products. The reaction mixture is then subjected to exonuclease digestion to degrade non-connected capturing probes and DNA sequences in the sample. The DNA ligase and exonuclease can be inactivated at 95° C. for 30 minutes.

### Amplification and Determination of CNV

Add universal PCR primers, two sequence-specific Taqman® probes, Taq DNA polymerase, dNTPs and suitable buffering components to the reaction mixture. The sequence-specific Taqman® probes recognizing the universal detection sequence on HER2-specific and RNase P-specific connection products contain a FAM and a VIC fluorophore, respectively. Perform a digital PCR on the reaction mixture and count the copy number of HER2 gene and the reference gene, RNase P by counting the number of chambers with FAM and VIC fluorescence, respectively. Determine the copy number variation by calculating the ratio between the copy number of HER2 and RNase P gene.

While the present invention has been described in some detail for purposes of clarity and understanding, one skilled in the art will appreciate that various changes in form and detail can be made without departing from the true scope of the invention, which is defined by the appended claims. All figures, tables, appendices, patents, patent applications and publications, referred to above, are hereby incorporated by reference.

What is claimed is:

1. A method for detecting a target sequence in a nucleic acid sample, comprising the steps of:
a) providing a target-capturing probe containing a target-specific portion and a universal primer binding and a universal detection portion;
b) Selecting a target-capturing probe from
i. a single polynucleotide, from 5′ to 3′ end sequentially comprising a 5′-target-specific sequence (5′-TSS), a forward universal primer binding sequence (Forward-UPBS), a breakable site, a reverse universal primer binding sequence (Reverse-UPBS) and a 3′-target-specific sequence (3′-TSS), having a universal detection sequence inserted between the Forward-UPBS and the 5′-TSS or the Reverse-UPBS and the 3′-TSS, or
ii. two polynucleotides, comprising a first polynucleotide that comprises an exonuclease-resistant 5′end, a Reverse-UPBS, and a 3′-TSS at the 3′ end, and a second polynucleotide that comprises a 5′-TSS at the 5′ end, a Forward-UPBS, and an exonuclease-resistant 3′ end, having a universal detection sequence inserted between the Forward-UPBS and the 5′-TSS or the Reverse-UPBS and the 3′-TSS;
c) contacting the target-capturing probe with the nucleic acid sample under conditions where the 3′-TSS and the 5′-TSS of the target-capturing probe anneal to complementary sequences in the nucleic acid sample to form a duplex nucleic acid;
d) performing a target-selective connection wherein the 3′-TSS and the 5′-TSS of the target-capturing probe are connected to form a linear or circular exonuclease-resistant target-specific connection product;
e) Optionally, using an exonuclease to digest susceptible nucleic acids, and after the enzymatic digestion, inactivating the exonuclease and/or purifying the exonuclease-resistant target-specific connection products;
f) breaking the circular exonuclease-resistant target-specific connection products, if present, at the breakable site to obtain linear target-specific connection products;
g) amplifying the linear target-specific connection products or the linear exonuclease-resistant target-specific connection products by a polymerase chain reaction (PCR) using universal PCR primers recognizing the universal primer binding sequences or the complement thereof; and
h) detecting or quantitating the linear target-specific connection products or the linear exonuclease-resistant target-specific connection products as a measurement of the target sequence using a sequence-specific reporter probe recognizing the universal detection sequence or the complement thereof.

2. The method of claim 1, wherein the target-capturing probe is used to capture a mutant variant of a target gene and functions as a mutant-capturing probe to carry out a mutant-selective connection to form a mutant-specific connection product in the step d).

3. The method of claim 2, further comprises repeating the step c) and d) multiple times for linear amplification of the mutant-specific connection product.

4. The method of claim 3, wherein the step c) and d) are repeated for 1-100 times.

5. The method of claim 2, wherein a blocking probe that is complementary to a sequence of a wild-type counterpart region of the mutated region in the mutant variant is added in step c), and wherein the blocking probe blocks the formation of an exonuclease-resistant connection product using the wild-type sequence as a template.

6. The method of claim 2, wherein a thermostable DNA ligase is used for the ligation of the 3′-TSS and the 5′-TSS.

7. The method of claim 2, wherein the exonuclease comprises one or more exonucleases that have 5′->3′ and 3′->5′ exonuclease activities and can digest single- and double-stranded DNAs.

8. The method of claim 2, wherein the amplification and the detection of the linear mutant-specific connection products or the linear exonuclease-resistant mutant-specific connection products happen concurrently.

9. The method of claim 2, wherein the sequence-specific reporter probe can give off a fluorescent signal once activated.

10. The method of claim 2, wherein the 3′-TSS and/or the 5′-TSS of the mutant-capturing probe anneal to a locus at or close to a mutant sequence of the target gene.

11. The method of claim 2, wherein the 3′-TSS and/or 5′-TSS of the mutant-capturing probe comprise a sequence complementary to a portion of the mutant sequence.

12. The method of claim 2, wherein the breakable site comprises a chemical moiety that is susceptible to photo, enzymatic or chemical cleavage.

13. The method of claim 2, wherein a plurality of mutant-capturing probes comprising different target-specific sequences and the same primer binding sequence and the same detection sequence are used to detect a plurality of mutant variants of target genes.

14. The method of claim 13, wherein the plurality of mutant-capturing probes comprise target-specific sequences complementary to different mutant variants of the same target gene.

15. The method of claim 13, wherein the plurality of mutant-capturing probes comprise target-specific sequences complementary to mutant variants of different target genes.

16. The method of claim 2, wherein a plurality of mutant-capturing probes are divided into multiple groups, wherein members of each group have different target-specific sequences, share a universal detection sequence that is specific for the group, and share the same universal primer binding sequences among all the members of different groups.

17. The method of claim 2, wherein mutations in the mutant variant compared to the wild-type sequence can be nucleotide substitution, deletion, insertion, gene fusion or any combination thereof.

18. The method of claim 2, wherein the 3′-TSS of the mutant-capturing probe contains a mutated nucleotide at its 3′ end and/or the 5′-TSS of the mutant-capturing probe contains a mutated nucleotide at its 5′ end.

19. The method of claim 18, wherein the 3′-TSS and the 5′-TSS of the mutant-capturing probe anneal adjacently at the mutated region of the mutant variant; and wherein the exonuclease-resistant mutant-specific connection product is formed by direct ligation of the 3′-TSS and the 5′-TSS.

20. The method of claim 2, wherein the 3′-TSS and the 5′-TSS of the mutant-capturing probe anneal to the target gene in the nucleic acid sample to form a DNA duplex with a single nucleotide gap at the locus of a mutated nucleotide; and wherein the exonuclease-resistant mutant-specific connection product is formed by extending the 3′-TSS with a single nucleotide complementary to the mutated nucleotide using a DNA polymerase and subsequent DNA ligation to connect the extended 3′-TSS and the 5′-TSS.

21. The method of claim 20, wherein the DNA polymerase is a thermostable DNA polymerase that lacks 5′->3′ exonuclease activity and does not express strand displacement activity.

22. The method of claim 2, wherein the 3′-TSS and the 5′-TSS of a mutant-capturing probe anneal to the target gene to form a DNA duplex with a multi-nucleotide gap, wherein the exonuclease-resistant mutant-specific connection product is formed by a polynucleotide extension from the 3′ end of the 3′-TSS using a DNA polymerase and subsequent DNA ligation to connect the extended 3′-TSS and the 5′-TSS.

23. The method of claim 22, wherein the 3′end of the 3′-TSS is complementary to a mutated nucleotide, and wherein DNA polymerases can only extend the 3′ end of the 3′-TSS using the mutant variant as a template.

24. The method of claim 23, wherein the DNA polymerase is a thermostable DNA polymerase that lacks 3′->5′ and 5′->3′ exonuclease activity and does not express strand displacement activity.

25. The method of claim 2, wherein the locus of a mutated region of the mutant variant is known but the identity of mutated nucleotides is unknown, wherein the 3′-TSS and the 5′-TSS are designed to be complementary to regions upstream and downstream of the mutated region, and wherein a non-extendable blocking probe that is complementary to the wild-type counterpart region of the mutated region is added in step c), wherein the non-extendable blocking probe blocks the annealing of the 3′-TSS and/or the 5′-TSS to the wild-type sequence, and/or extension of the 3′-TSS by DNA polymerases using the wild-type sequence as a template.

26. The method of claim 25, wherein the sequence of the non-extendable blocking probe overlaps with the 3′-TSS and/or 5′-TSS.

27. The method of claim 25, wherein the non-extendable blocking probe comprises modified nucleotides.

28. The method of claim 27, wherein the modified nucleotide can be a peptide nucleic acid, a locked nucleic acid, or a nucleotide linked to a minor groove binder.

29. The method of claim 2, wherein the nucleic acid sample is an RNA sample and wherein the 3′-TSS and the 5′-TSS are connected by a RNA-templated DNA ligation.

30. The method of claim 29, wherein the extension of the 3′-TSS using an RNA template is carried out by use of a reverse transcriptase.

31. The method of claim 2, wherein the method is used to detect the presence of methylation of the target gene, wherein the target gene is modified by converting all the non-methylated cytosine to uracil while the methylated cytosine remains unchanged as a cytosine, wherein the detection of the methylation of the target gene is to detect the presence of the U->C mutation at a putative methylation site in the modified target gene.

32. The method of claim 2, wherein the method is used to detect a gene fusion mutant having a first gene portion, a fusion locus, and a second gene portion.

33. The method of claim 32, wherein the 3′-TSS of the mutant-capturing probe is complementary to a region of the first gene and the 5′-TSS of the mutant-capturing probe is complementary to a region of the second gene.

34. The method of claim 32, wherein the 3′-TSS comprises a sequence complementary to a region having portions of the first and the second gene, and the 5′-TSS comprises a sequence complementary to a region of the second gene.

35. The method of claim 32, wherein the 3′-TSS comprises a sequence complementary to a region of the first gene and the 5′-TSS comprises a sequence complementary to a region having portions of the first and the second gene.

36. The method of claim 32, wherein the 3′-TSS and the 5′-TSS adjacently anneal to the gene fusion mutant.

37. The method of claim 32, wherein the 3′-TSS and the 5′-TSS anneal to the gene fusion mutant to form a duplex with a single- or multi-nucleotide gap.

38. The method of claim 32, wherein a blocking probe complementary to a wild-type sequence encompassing the gene fusion locus is added in step c).

39. The method of claim 32, wherein the gene fusion mutant has a known first gene portion, a known fusion locus, and an unknown second gene portion.

40. The method of claim 39, wherein the 3′-TSS comprises a sequence complementary to a region in the first gene and the 5′-TSS comprises a sequence complementary to a predesigned sequence tag that is ligated to the unknown second gene, and wherein a blocking probe complementary to a wild-type sequence encompassing the gene fusion locus is added in step c).

41. The method of claim 1, wherein the target-capturing probe is used to detect a wild-type sequence of a target gene.

42. The method of claim 41, wherein the method is used to detect copy number variation of a target gene/chromosome, wherein a plurality of target-capturing probes are used to detect multiple gene loci of the target gene/chromosome and those of a reference gene/chromosome.

43. The method of claim 42, wherein the plurality of target-capturing probes are divided into two groups, each having distinct detection sequences and being specific for sequences of the target and the reference gene/chromosome, respectively.

44. A kit for detecting a mutant variant of a target gene in a nucleic acid sample containing an excess of wild-type sequences, comprising:
a) a mutant-capturing probe containing a target-specific portion and a universal primer binding and a universal detection portion, comprising
i. a single polynucleotide, from 5′ to 3′ end sequentially comprising a 5′-target-specific sequence (5′-TSS), a forward universal primer binding sequence (Forward-UPBS), a breakable site, a reverse universal primer binding sequence (Reverse-UPBS) and a 3′-target-specific sequence (3′-TSS), having a universal detection sequence inserted between the Forward-UPBS and the 5′-TSS or the Reverse-UPBS and the 3′-TSS or
ii. two polynucleotides, comprising a first polynucleotide that comprises an exonuclease-resistant 5′end, a Reverse-UPBS, and a 3′-TSS at the 3′ end, and a second polynucleotide that comprises a 5′-TSS at the 5′ end, a Forward-UPBS, and an exonuclease-resistant 3′ end, having a universal detection sequence inserted between the Forward-UPBS and the 5′-TSS or the Reverse-UPBS and the 3′-TSS;
b) a DNA ligase;
c) a sequence-specific reporter probe recognizing the universal detection sequence;
d) an exonuclease;
e) optionally a DNA polymerase; and
f) an instruction manual.

45. The kit of claim 44, wherein the 3′-TSS contains a mutated nucleotide at its 3′ end and/or the 5′-TSS contains a mutated nucleotide at its 5′ end, and the 3′-TSS and the 5′-TSS anneal adjacently at the mutated region of the mutant variant.

46. The kit of claim 44, wherein the 3′-TSS and the 5′-TSS anneal to the target gene to form a DNA duplex with a single nucleotide gap, wherein the single nucleotide gap is at the locus of a mutated nucleotide.

47. The kit of claim 44, wherein the 3′-TSS and the 5′-TSS anneal to the target gene to form a DNA duplex with a multi-nucleotide, wherein the multi-nucleotide gap overlaps with a mutated region.

48. The kit of claim 44, further comprises a non-extendable blocking probe comprising a sequence complementary to wild-type counterpart sequence of the mutated sequence in the mutant variant.

49. The kit of claim 44, comprising a plurality of mutant-capturing probes, each having a pair of TSS complementary to regions of respective target gene sequence close to or at a mutated region, and wherein all the mutant-capturing probes have the same Forward-UPBS and Reverse-UPBS and the same detection sequence.

50. A kit for detecting and quantitating a target gene in a nucleic acid sample, comprising:
a) a target-capturing probe containing a target-specific portion and a universal primer binding and a universal detection portion, comprising
i. a single polynucleotide, from 5′ to 3′ end sequentially comprising a 5′-TSS, a Forward-UPBS, a breakable site, a Reverse-UPBS and a 3′-TSS, having a universal detection sequence inserted between the Forward-UPBS and the 5′-TSS or the Reverse-UPBS and the 3′-TSS, or
ii. two polynucleotides, comprising a first polynucleotide that comprises an exonuclease-resistant 5′ end, a Reverse-UPBS, and a 3′-TSS at the 3′ end, and a second polynucleotide that comprises a 5′-TSS at the 5′ end, a Forward-UPBS, and an exonuclease-resistant 3′ end, having a universal detection sequence inserted between the Forward-UPBS and the 5′-TSS or the Reverse-UPBS and the 3′-TSS;
b) a DNA ligase;
c) a reporter probe specific for the detection sequence;
d) an exonuclease;
e) optionally a DNA polymerase; and
f) an instruction manual.

51. The kit of claim 50, wherein the kit comprises a plurality of target-capturing probes, each having a pair of target-specific sequences complementary to regions of respective target gene sequences, wherein all the target-capturing probes have the same Forward-UPBS and Reverse-UPBS and the same universal detection sequence.

52. The kit of claim 51, wherein the plurality of target-capturing probes comprise target-specific sequences complementary to different regions of a target gene or a target chromosome.

53. The kit of claim 50, wherein a plurality of target-capturing probes are divided into multiple groups, each group having a distinct universal detection sequence coupled to a different detection signal.

54. The kit of claim 53, wherein the plurality of target-capturing probes are divided into a first and a second group, wherein the target-capturing probes of the first group are specific for multiple gene loci on a target gene/chromosome, and the target-capturing probes of a second group are specific for multiple gene loci on a reference gene/chromosome, and wherein the kit is used for detection of copy number variation of the target gene/chromosome.
